(19) United States
(12) Patent Application Publication
(10) Pub. No.: US 2021/0401887 A1
(43) Pub. Date: Dec. 30, 2021

(54) T CELLS FROM LYMPHATIC FLUID FOR DIAGNOSTIC AND THERAPEUTIC USE

(71) Applicant: THE CHILDREN'S HOSPITAL OF PHILADELPHIA, Philadelphia, PA (US)

(72) Inventors: David BARRETT, Philadelphia, PA (US); Yoav DORI, Philadelphia, PA (US)

(73) Assignee: THE CHILDREN'S HOSPITAL OF PHILADELPHIA, Philadelphia, PA (US)

(21) Appl. No.: 17/289,877

(22) PCT Filed: Oct. 30, 2019
(86) PCT No.: PCT/US19/58721

§ 371 Date: Apr. 29, 2021

Related U.S. Application Data
(60) Provisional application No. 62/753,613, filed on Oct. 31, 2018.
(60) Provisional application No. 62/827,562, filed on Apr. 1, 2019.

(51) Int. Cl. A61K 35/17; C12N 5/0783; A61P 35/02; A61P 35/00; A61K 45/06; A61K 39/00; A61K 39/39; C07K 16/28; C07K 14/725; C07K 14/705; A61K 38/17; A61K 39/395

(52) U.S. Cl. CPC A61K 35/17; A61K 2039/505; A61P 35/02; A61P 35/00; A61K 45/06; A61K 39/0011; A61K 39/39; C07K 16/2803; C07K 14/7051; C07K 14/70578; A61K 38/1774; A61K 38/177; A61K 39/3955; C12N 2510/00; A61K 2039/5156; A61K 2039/5158; A61K 2039/545; A61K 2039/54; A61K 2039/55516; C07K 2317/76; C07K 2319/30; C07K 2319/33; C12N 5/0636

(57) ABSTRACT

The present disclosure provides improved compositions and methods for T-cell-based immunotherapy employing a modified T cells obtained from the lymphatic system of normal and cancer patient donors. These cells can then be used to treat patients for a variety of different blood and solid tumor cancers.

### PRIORITY CLAIM

This application claims benefit of priority to U.S. Provisional Application Ser. No. 62/753,613, filed Oct. 31, 2019, and U.S. Provisional Application Ser. No. 62/827,562, filed Apr. 1, 2019, the entire contents of both applications being hereby incorporated by reference.

### BACKGROUND

### 1. Technical Field

The present disclosure relates generally to the fields of medicine, immunology, cell biology, and molecular biology. In certain aspects, the field of the disclosure concerns adoptive immunotherapy. More particularly, it unique populations of T-cells obtained from the lymphatic system of a subject, in particular a subject with cancer, and the use of such cells for diagnostic and therapeutic methods.

### 2. Background

Adoptive immunotherapy is used to help the immune system fight diseases, such as cancer and infections with certain viruses. T cells are collected from a patient and expanded ex vivo, increasing the number of T cells that are able to kill cancer cells or tight infections. These T cells are given back to the patient to help the immune system fight disease. The T cells can also be transduced with genetic material, such as expression constructs encoding engineered alpha and beta chains of the TCR, a single chain variable fragment (scFv) of an antibody, fused to a transmembrane domain and intracellular domains containing signaling molecules or modules, to specifically recognize a cell surface antigen on a target cell type of choice. Such chimeric antigen receptor (CAR)-T cells targeting tumor-associated antigens have shown promise in the treatment of some malignancies, most notably Pre-B acute lymphoblastic leukemia.

Lymphatic fluid circulates through a separate vascular system before emptying into the venous circulation in the chest. T cells make up a large majority of the cells in lymphatic fluid, but little is known about their functional status. Cellular therapies such CAR-T therapy depend on collecting large numbers of Naïve or early memory T cells, which may not be available in peripheral blood. Thus, new sources of T cells, including those that have characteristics that improve their utility in the development and application of cell-based immunotherapies are needed.

### SUMMARY

Thus, in accordance with the present disclosure, there is provided a method of preparing a T cell comprising (a) obtaining a T cell-containing sample from the lymphatic system of a subject; (b) isolating a T cell subpopulation from the sample; and (c) generating a chimeric antigen receptor (CAR) T cell from the isolated T cell subpopulation of step (b). Step (b) may comprise isolating a cell based on CD3/28 expression.

The T cell subpopulation isolated in step (b) may have has reduced levels of negative checkpoint regulators (NCR) as compared to the average T cell NCR isolated from peripheral blood of the subject, and/or has increased expression of GLUT-1 as compared to the average T cell in the sample. The NCR may be PD-1, LAG3 or Tim3. The T cell subpopulation isolated in step (b) may be enhanced in Naïve T cell and/or a Stem Cell Memory (SCM) T cell content as compared to an unisolated population.

The subject may be a disease-free subject, may be diseased but does not have cancer, such as where the subject has a congenital or acquired lymphatic malformation or chylothorax. The subject may have cancer, such as a solid cancer selected from lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, brain cancer, thyroid cancer, various types of head and neck cancer, or melanoma, or a a blood cancer such as leukemia or lymphoma.

The subject may not have been subjected to chemotherapy or radiotherapy, or have been subjected to chemotherapy or radiotherapy, or have been subjected to chemotherapy and radiotherapy. The subject may be a human subject.

In another embodiment, there is provided method of treating cancer in a human subject in need thereof comprising administering to the subject an effective amount of a cell therapy comprising one or more cells produced as described above. The method may further comprise administering to the human subject a second cancer therapy, such as chemotherapy, immunotherapy, radiotherapy, hormone therapy or surgery. The second cancer therapy may be administered at the same time as the cell therapy or administered before or after the cell therapy. The method may further comprise administering to the human subject a second administration of an effective amount of one or more cells produced as described above.

The cancer may be a metastatic, recurrent or drug-resistant cancer. The cell therapy may be administered local to cancer site, region to a cancer site, or systemically. The cancer may be solid cancer such as lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, brain cancer, vulval cancer, thyroid cancer, various types of head and neck cancer, and melanoma. The cancer may be a blood cancer such as leukemia or lymphoma.

Also provided is a vaccine composition comprising a cell produced as described above. The vaccine composition may further comprise an adjuvant, may further comprise a biological response modifier, and/or ma further comprise a chemokine. Two distinct CAR T cells with different binding specificities may be comprised in the vaccine composition.

In an additional embodiment, there is provided a method of generating an anti-cancer immune response is a subject comprising administering to the subject a vaccine composition as described above.

Other objects, features and advantages of the present disclosure will become apparent from the following detailed description. It should be understood, however, that the detailed description and the specific examples, while indicating particular embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1—Different paths of T cell stimulation in normal versus cancer patients.

FIGS. 2A-D—(FIG. 2A) T cells from ALL and NHL patients were subjected to CAR manufacture conditions, Y-axis represents percentage of samples that would have passed clinical criteria for successful manufacture. T cells from leukemia patients have a high potential from diagnosis but lose this with cumulative chemotherapy whereas NHL T cells have poor potential even prior to chemotherapy. (FIG. 2B) Expression of negative checkpoint regulators on circulating T cells at diagnosis. Solid tumors and lymphomas are notable for an increased number of T cells expressing more than one NCR compared to leukemia patients. (FIG. 2C) There is no correlation between the absolute T cell count (ATC) and CAR manufacture potential. (FIG. 2D) Expansion potential at diagnosis of pediatric cancer patient T cells. Red line and below is failure, Green line and above passing and in between is a grey zone where some potential may remain.

FIGS. 3A-B—(FIG. 3A) Effects of chemotherapy agents at anti-tumor dosing on mature T cells. Red line indicates baseline apoptosis of T cells in culture with 20 U/mL of IL-2 over 24 hours. T cells were either left in the IL-2 or stimulated simultaneously with CD3/28 beads as in CAR manufacture. Cisplatin, cyclophosphamide, and doxorubicin are the most detrimental to mature T cells in this time frame. (FIG. 3B) As the inventors have discovered, Naïve and memory T cells display differential sensitivity to chemotherapy. Doxorubicin induces GLUT-1 expression and the subsequent increased uptake of glucose with stimulation. Memory T cells, with existing high GLUT-1, are not significantly affected.

FIGS. 4A-C—(FIG. 4A) Oxygen Consumption Rate (OCR) from normal donor T cells either unsorted or sorted into purified subsets as indicated. Peak OCR is reflective of the spare respiratory capacity (SRC) of the mitochondria of the T cells. SRC is correlated with T cell proliferative capacity and efficacy. Bulk T cells are most notably affected by cyclophosphamide and mitoxantrone which largely abolishes the SRC of T cells. Cytarabine and doxorubicin do not appear to have much effect until T cells are sorted. Naive T cells do show significant SRC reduction with doxorubicin, whereas the memory T cells are less affected. This may be due to the apparent reliance of T cells exposed to doxorubicin on glycolysis (see next section), and that memory T cells already have this pathway activated and thus may be resistant to doxorubicin mediated effects. (FIG. 4B) Mitochondrial mass as measured by the Mitotracker Green dye. (upper panel) and membrane potential as measured by the JC1 dye ratio (lower panel). Cyclophosphamide significantly alters mitochondrial architecture and membrane potential. (FIG. 4C) Addition of palmitate to the media of T cells exposed to chemotherapy helps restore some SRC, and this appears specific based on inhibiton of this effect by etoximir.

FIG. 5—Nanostring RNA analysis of genes in metabolic pathways on T cells from pre-chemotherapy patient samples (feft). Results were subjected to unsupervised clustering, with correlated with a separate assessment of CAR T cell potential (expansion with CD3/28 beads). ALL and Wilms tumor patients resemble T cells from normal donors whereas lymphoma and solid tumor patients cluster together. ALL #2, which passed pre-chemotherapy, demonstrated cumulative changes in metabolic gene expression with each cycle of chemotherapy (right).

FIG. 6—Collection of lymphatic fluid from the thoracic duct. First, a lymph node is identified in the groin under ultrasound guidance, and contrast is injected into the node. The thoracic duct is identified under fluoroscopy, and a Chiba needle is inserted into the abdomen into the thoracic duct to collect specimen.

FIGS. 7A-C—Characteristics of T cells isolated from lymphatic duct compared to blood. (FIG. 7A) Percent of T cell subsets determined by flow cytometry. (FIG. 7B) Percentage of T cells expressing negative checkpoint regulators PD1, Tim3, and Lag3, determined by flow cytometry. (FIG. 7C) Expansion of T cells from blood and lymphatic when stimulated with CD3/28 beads with or without cytokines. *p<0.05, ****p<0.0001

FIGS. 8A-D—Generation of CAR T cells from lymphatic fluid and blood for in vivo mouse modeling. (FIG. 8A) Blood and lymphatic fluid from the same patient was stimulated with CD3/28 beads and transduced with lentivirus encoding CD19 CAR. Graphs represent cell count and size measured over 14 days. Data is a representative example of 5 repeated experiments. (FIG. 8B) CD8 and CD 4 composition of manufactured CAR T cells by flow cytometry. (FIG. 8C) CAR protein expression on surface of T cells after manufacturing by flow cytometry. (FIG. 8D) Murine leukemia model treated with CAR T cells generated from blood and lymphatic fluid. Bioluminescent leukemia cells measured in the mice as mean radiance over time. ****p<0.0001

### DESCRIPTION OF ILLUSTRATIVE EMBODIMENTS

While immunotherapies including adoptive T cell therapy has shown remarkable recent results, there remain limitations, including proper sourcing of T cells with optimal characteristics for expansion and therapeutic administration. The inventors characterized peripheral blood and lymphatic fluid T cells from a variant of normal, non-cancer diseased, and cancer-affected patients receiving therapeutic thoracic duct access procedures and identified the phenotype and CAR-T cell potential. The shift in naïve and SCM T cells in lymphatic fluid versus peripheral blood suggest that lymphatic fluid is rich in cells that are potentially better suited to CAR-T manufacture, raising the possibility that collecting cells from this source rather than peripheral blood is preferred.

Lymphatic fluid in the thoracic duct was rich in T cells, with higher percentage of naïve and stem central memory T cell subsets compared to paired blood samples. T cells from lymphatic fluid showed decreased negative checkpoint regulators on the surface and increased rapid expansion with bead activation. Creation of CD19-directed CAR T cells from blood and lymphatic T cells showed similar lentiviral transduction properties, but CAR T cells generated from lymphatic fluid produced markedly superior cytotoxicity in a murine leukemia model. These results are the first characterization of T cells from the thoracic duct of pediatric patients and suggest an alternative approach for manufacturing of cellular therapy that will improve both expansion and cytotoxic effect. These and other aspects of the disclosure are set out below.

### I. DEFINITIONS

In this disclosure, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements and components comprising one unit and elements or components that comprise more than one unit unless specifically stated otherwise.

As used herein, the term “about,” when used in conjunction with a percentage or other numerical amount, means plus or minus 10% of that percentage or other numerical amount. For example, the term “about 80%,” would encompass 80% plus or minus 8%.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials define a term in a manner that contradicts the definition of that term in this application, this application controls.

As used herein, and unless otherwise indicated, the terms “disease”, “disorder” or “condition” refer to a state of being or health status of a patient or subject capable of being treated with a compound, pharmaceutical composition, or method provided herein. In some embodiments, the disease is cancer (e.g. pancreatic cancer, colon cancer, gastric cancer, lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma, gastric cancer).

As used herein, and unless otherwise indicated, the terms “treating”, or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, include prevention of an injury, pathology, condition, or disease. In some embodiments, “treating” refers to the treatment of cancer.

As used herein, and unless otherwise indicated, the terms “prevent,” “preventing,” and “prevention” contemplate an action that occurs before a patient begins to suffer from a disorder that involves cancer that delays the onset of, and/or inhibits or reduces the severity of cancer.

As used herein, and unless otherwise indicated, the terms “manage,” “managing,” and “management” encompass preventing, delaying, or reducing the severity of a recurrence of a disorder such as cancer in a patient who has already suffered from such a disease, disorder or condition. The terms encompass modulating the threshold, development, and/or duration of the disorder that involves cancer or changing how a patient responds to the disorder that involves cancer.

As used herein, and unless otherwise specified, a “therapeutically effective amount” of a compound is an amount sufficient to provide any therapeutic benefit in the treatment or management of a disorder that involves electrically active cells, such as but not limited to neuronal dysfunction, a neuron mediated disorder, ocular disorder or cardiac disorder, or to delay or minimize one or more symptoms associated with a disorder that involves electrically active cells, such as but not limited to neuronal dysfunction, a neuron mediated disorder, ocular disorder or cardiac disorder. A therapeutically effective amount of a compound means an amount of the compound, alone or in combination with one or more other therapies and/or therapeutic agents that provide any therapeutic benefit in the treatment or management of a disorder that involves electrically active cells, such as but not limited to neuronal dysfunction, a neuron mediated disorder, ocular disorder or cardiac disorder.

As used herein, and unless otherwise specified, an “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of a “therapeutically effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing the severity or frequency of the symptom(s), or elimination of the symptom(s). The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, and unless otherwise specified, a “prophylactically effective amount” of a compound is an amount sufficient to prevent or delay the onset of cancer or one or more symptoms associated with cancer or prevent or delay its recurrence. A prophylactically effective amount of a compound means an amount of the compound, alone or in combination with one or more other treatment and/or prophylactic agent that provides a prophylactic benefit in the prevention of a disorder such as cancer. The term “prophylactically effective amount” can encompass an amount that prevents a disorder such as cancer, improves overall prophylaxis, or enhances the prophylactic efficacy of another prophylactic agent. The “prophylactically effective amount” can be prescribed prior to, for example, the development of a disorder such as cancer.

As used herein, “patient” or “subject in need thereof” refers to a living organism suffering from or prone to a disease or condition that can be treated by administration of a composition or pharmaceutical composition as provided herein. Non-limiting examples include humans, primates, companion animals (dogs, cats, etc.), other mammals, such as but not limited to, bovines, rats, mice, monkeys, goat, sheep, cows, deer, as well as other non-mammalian animals. In some embodiments, a patient is human.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY, 2nd ed., J. Wiley & Sons (New York, N.Y. 1994); Sambrook et al., MOLECULAR CLONING, A LABORATORY MANUAL, Cold Springs Harbor Press (Cold Springs Harbor, N Y 1989). Any methods, devices, and materials similar or equivalent to those described herein can be used in the practice of this disclosure. The following definitions are provided to facilitate understanding of certain terms used frequently herein and are not meant to limit the scope of the present disclosure.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “cell” as used herein, refers to a cell carrying out metabolic or other functions sufficient to preserve or replicate its genomic DNA. A cell can be identified by well-known methods in the art including, for example, the presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example, mammalian, insect (e.g., Spodoptera) and human cells. Cells may be useful when they are naturally non-adherent or have been treated not to adhere to surfaces, for example by trypsinization.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

“Nucleic acid” refers to deoxyribonucleotides or ribonucleotides and polymers thereof, in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single- and double-stranded DNA, single- and double-stranded RNA (including siRNA), and hybrid molecules having mixtures of single- and double-stranded DNA and RNA. Nucleic acid as used herein also refers to nucleic acids that have the same basic chemical structure as a naturally occurring nucleic acid. Such analogs have modified sugars and/or modified ring substituents but retain the same basic chemical structure as the naturally occurring nucleic acid. A nucleic acid mimetic refers to chemical compounds that have a structure that is different the general chemical structure of a nucleic acid, but that functions in a manner similar to a naturally occurring nucleic acid. Examples of such analogs include, without limitation, phosphorothioates, phosphoramidites, methyl phosphonates, chiral-methyl phosphonates, 2-O-methyl ribonucleotides, and peptide-nucleic acids (PNAs).

The word “expression” or “expressed” as used herein in reference to a gene means the transcriptional and/or translational product of that gene. The level of expression of a DNA molecule in a cell may be determined on the basis of either the amount of the corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 MOLECULAR CLONING: A LABORATORY MANUAL, 18.1-18.88. Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selective advantage to the transfected cell. Such a selective advantage may be a resistance towards a certain toxin that is presented to the cell. Expression of a transfected gene can further be accomplished by transposon-mediated insertion into to the host genome. During transposon-mediated insertion, the gene is positioned in a predictable manner between two transposon linker sequences that allow insertion into the host genome as well as subsequent excision. Stable expression of a transfected gene can further be accomplished by infecting a cell with a lentiviral vector, which after infection forms part of (integrates into) the cellular genome thereby resulting in stable expression of the gene.

The terms “plasmid”, “vector” or “expression vector” refer to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

The terms “transfection”, “transduction”, “transfecting” or “transducing” can be used interchangeably and are defined as a process of introducing a nucleic acid molecule or a protein to a cell. Nucleic acids are introduced into a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetization and electroporation. In some embodiments, the nucleic acid molecules are introduced into a cell using electroporation following standard procedures are well known in the art. For viral-based methods of transfection, any useful viral vector may be used in the methods described herein. Examples of viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) and Prochiantz (2007).

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high-performance liquid chromatography. A protein that is the predominant species present in a preparation is substantially purified.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

“Anti-cancer agent” is used in accordance with its plain ordinary meaning and refers to a composition (e.g., compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer.

### II. LYMPHATIC SYSTEM

The lymphatic system is part of the vascular system and an important part of the immune system, comprising a network of lymphatic vessels that carry a clear fluid called lymph directionally towards the heart. Unlike the circulatory system, the lymphatic system is not a closed system. The human circulatory system processes an average of 20 liters of blood per day through capillary filtration, which removes plasma while leaving the blood cells. Roughly 17 litres of the filtered plasma is reabsorbed directly into the blood vessels, while the remaining three litres remain in the interstitial fluid. One of the main functions of the lymph system is to provide an accessory return route to the blood for the surplus three liters.

The other main function is that of defense in the immune system. Lymph is very similar to blood plasma: it contains lymphocytes. It also contains waste products and cellular debris together with bacteria and proteins. Associated organs composed of lymphoid tissue are the sites of lymphocyte production. Lymphocytes are concentrated in the lymph nodes. The spleen and the thymus are also lymphoid organs of the immune system. The tonsils are lymphoid organs that are also associated with the digestive system. Lymphoid tissues contain lymphocytes, and also contain other types of cells for support. The system also includes all the structures dedicated to the circulation and production of lymphocytes (the primary cellular component of lymph), which also includes the bone marrow, and the lymphoid tissue associated with the digestive system.

The blood does not come into direct contact with the parenchymal cells and tissues in the body (except in case of an injury causing rupture of one or more blood vessels), but constituents of the blood first exit the microvascular exchange blood vessels to become interstitial fluid, which comes into contact with the parenchymal cells of the body. Lymph is the fluid that is formed when interstitial fluid enters the initial lymphatic vessels of the lymphatic system. The lymph is then moved along the lymphatic vessel network by either intrinsic contractions of the lymphatic passages or by extrinsic compression of the lymphatic vessels via external tissue forces (e.g., the contractions of skeletal muscles), or by lymph hearts in some animals. The organization of lymph nodes and drainage follows the organization of the body into external and internal regions; therefore, the lymphatic drainage of the head, limbs, and body cavity walls follows an external route, and the lymphatic drainage of the thorax, abdomen, and pelvic cavities follows an internal route. Eventually, the lymph vessels empty into the lymphatic ducts, which drain into one of the two subclavian veins, near their junction with the internal jugular veins.

The lymphatic system plays a major role in the body's immune system, as the primary site for cells relating to adaptive immune system including T-cells and B-cells. Cells in the lymphatic system react to antigens presented or found by the cells directly or by other dendritic cells. When an antigen is recognized, an immunological cascade begins involving the activation and recruitment of more and more cells, the production of antibodies and cytokines and the recruitment of other immunological cells such as macrophages.

The lymphatic system consists of lymphatic organs, a conducting network of lymphatic vessels, and the circulating lymph. The primary or central lymphoid organs generate lymphocytes from immature progenitor cells. The thymus and the bone marrow constitute the primary lymphoid organs involved in the production and early clonal selection of lymphocyte tissues. Bone marrow is responsible for both the creation of T cells and the production and maturation of B cells. From the bone marrow, B cells immediately join the circulatory system and travel to secondary lymphoid organs in search of pathogens. T cells, on the other hand, travel from the bone marrow to the thymus, where they develop further. Mature T cells join B cells in search of pathogens. The other 95% of T cells begin a process of apoptosis, a form of programmed cell death.

Secondary or peripheral lymphoid organs, which include lymph nodes and the spleen, maintain mature naïve lymphocytes and initiate an adaptive immune response. The peripheral lymphoid organs are the sites of lymphocyte activation by antigens. Activation leads to clonal expansion and affinity maturation. Mature lymphocytes recirculate between the blood and the peripheral lymphoid organs until they encounter their specific antigen.

Secondary lymphoid tissue provides the environment for the foreign or altered native molecules (antigens) to interact with the lymphocytes. It is exemplified by the lymph nodes, and the lymphoid follicles in tonsils, Peyer's patches, spleen, adenoids, skin, etc., that are associated with the mucosa-associated lymphoid tissue (MALT). In the gastrointestinal wall the appendix has mucosa resembling that of the colon, but here it is heavily infiltrated with lymphocytes.

The tertiary lymphoid tissue typically contains far fewer lymphocytes and assumes an immune role only when challenged with antigens that result in inflammation. It achieves this by importing the lymphocytes from blood and lymph.

The lymphatic vessels, also called lymph vessels, conduct lymph between different parts of the body. They include the tubular vessels of the lymph capillaries, and the larger collecting vessels—the right lymphatic duct and the thoracic duct (the left lymphatic duct). The lymph capillaries are mainly responsible for the absorption of interstitial fluid from the tissues, while lymph vessels propel the absorbed fluid forward into the larger collecting ducts, where it ultimately returns to the bloodstream via one of the subclavian veins. These vessels are also called the lymphatic channels or simply lymphatics.

The lymphatics are responsible for maintaining the balance of the body fluids. Its network of capillaries and collecting lymphatic vessels work to efficiently drain and transport extravasated fluid, along with proteins and antigens, back to the circulatory system.

Numerous intraluminal valves in the vessels ensure a unidirectional flow of lymph without reflux. Two valve systems are used to achieve this one directional flow—a primary and a secondary valve system. The capillaries are blind-ended, and the valves at the ends of capillaries use specialized junctions together with anchoring filaments to allow a unidirectional flow to the primary vessels. The collecting lymphatics, however, act to propel the lymph by the combined actions of the intraluminal valves and lymphatic muscle cells.

### III. HOST LYMPHATIC CELLS AND ENGINEERING OF THE SAME

A. Host Cells

Certain embodiments of the present disclosure concern host immune cells which can be engineered to express a chimeric antigen receptor (CAR). The immune cells may be T cells (e.g., memory T cells, Naïve T cells, regulatory T cells, CD4+ T cells, CD8+ T cells, or gamma-delta T cells), Natural Killer (NK) cells, invariant NK cells, or NKT cells. Also provided herein are methods of producing and engineering the immune cells as well as methods of using and administering the cells for adoptive cell therapy, in which case the cells may be autologous or allogeneic. Thus, the immune cells may be used as immunotherapy, such as to target cancer cells.

The immune cells may be isolated from subjects, particularly human subjects. The immune cells can be obtained from a subject of interest, such as a subject suspected of having a particular disease or condition, a subject suspected of having a predisposition to a particular disease or condition, a subject who is undergoing therapy for a particular disease or condition, such as cancer, a subject who is a healthy volunteer or healthy donor, or from blood bank. The isolated immune cells may be used directly, or they can be stored for a period of time, such as by freezing.

The immune cells may be enriched/purified. In some aspects, immune cells have enhanced immunomodulation capacity, such as measured by CD4- or CD8-positive T cell suppression. In specific aspects, the immune cells are isolated from pooled sources, for enhanced immunomodulation capacity. The pooled sources may be from 2 or more sources, such as 3, 4, 5, 6, 7, 8, 9, 10 or more sources (e.g., donor subjects). In other aspects, the immune cells have reduced levels of negative checkpoint regulators.

The population of immune cells can be obtained from a subject in need of therapy or suffering from a disease. Thus, the cells will be autologous to the subject in need of therapy. Alternatively, the population of immune cells can be obtained from a donor, such as a histocompatibility matched donor. The immune cells can be isolated from a pool of subjects and/or donors, such as from pooled cord blood.

When the population of immune cells is obtained from a donor distinct from the subject, the donor may be allogeneic, provided the cells obtained are subject-compatible in that they can be introduced into the subject. Allogeneic donor cells are may or may not be human-leukocyte-antigen (HLA)-compatible. To be rendered subject-compatible, allogeneic cells can be treated to reduce immunogenicity.

1. T Cells

In some embodiments, the immune cells are T cells. Several basic approaches for the derivation, activation and expansion of functional anti-tumor effector cells have been described in the last two decades. These include: autologous cells, such as tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. These approaches have given rise to numerous protocols for T cell preparation and immunization which can be used in the methods described herein.

In some embodiments, the T cells are derived from the blood, bone marrow, lymph, umbilical cord, or lymphoid organs. In some aspects, the cells are human cells. The cells typically are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells (e.g., CD4⁺ and/or CD8⁺ T cells) are Naïve T (T_{N}) cells, effector T cells (T_{EFF}), memory T cells and sub-types thereof, such as stem cell memory T (TSC_{M}), central memory T (TC_{M}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, one or more of the T cell populations is enriched for or depleted of cells that are positive for a specific marker, such as surface markers, or that are negative for a specific marker. In some cases, such markers are those that are absent or expressed at relatively low levels on certain populations of T cells (e.g., non-memory cells) but are present or expressed at relatively higher levels on certain other populations of T cells (e.g., memory cells).

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naïve, memory, and/or effector T cell subpopulations.

In some embodiments, CD8⁺ T cells are further enriched for or depleted of naive, central memory, effector memory, and/or central memory stem cells, such as by positive or negative selection based on surface antigens associated with the respective subpopulation. In some embodiments, enrichment for central memory T (TC_{M}) cells is carried out to increase efficacy, such as to improve long-term survival, expansion, and/or engraftment following administration, which in some aspects is particularly robust in such sub-populations. See Terakura et al. (2012); Wang et al. (2012).

In some embodiments, the T cells are autologous T cells. In this method, tumor samples are obtained from patients and a single cell suspension is obtained. The single cell suspension can be obtained in any suitable manner, e.g., mechanically (disaggregating the tumor using, e.g., a gentleMACS™ Dissociator, Miltenyi Biotec, Auburn, Calif.) or enzymatically (e.g., collagenase or DNase). Single-cell suspensions of tumor enzymatic digests are cultured in interleukin-2 (IL-2). The cells are cultured until confluence (e.g., about 2×10⁶ lymphocytes), e.g., from about 5 to about 21 days, such as from about 10 to about 14 days. For example, the cells may be cultured from 5 days, 5.5 days, or 5.8 days to 21 days, 21.5 days, or 21.8 days, such as from 10 days, 10.5 days, or 10.8 days to 14 days, 14.5 days, or 14.8 days.

The cultured T cells can be pooled and rapidly expanded. Rapid expansion provides an increase in the number of antigen-specific T-cells of at least about 50-fold (e.g., 50-, 60-, 70-, 80-, 90-, or 100-fold, or greater) over a period of about 10 to about 14 days. Rapid expansion can provide an increase of at least about 200-fold (e.g., 200-, 300-, 400-, 500-, 600-, 700-, 800-, 900-, or greater) over a period of about 10 to about 14 days.

Expansion can be accomplished by any of a number of methods as are known in the art. For example, T cells can be rapidly expanded using non-specific T-cell receptor stimulation in the presence of feeder lymphocytes and either interleukin-2 (IL-2) or interleukin-15 (IL-15), with IL-2 being particularly contemplated. The non-specific T-cell receptor stimulus can include around 30 ng/ml of OKT3, a mouse monoclonal anti-CD3 antibody (available from Ortho-McNeil®, Raritan, N.J.). Alternatively, T cells can be rapidly expanded by stimulation of peripheral blood mononuclear cells (PBMC) in vitro with one or more antigens (including antigenic portions thereof, such as epitope(s), or a cell) of the cancer, which can be optionally expressed from a vector, such as a human leukocyte antigen A2 (HLA-A2) binding peptide, in the presence of a T-cell growth factor, such as 300 IU/ml IL-2 or IL-15, with IL-2 being contemplated. The in vitro-induced T-cells are rapidly expanded by re-stimulation with the same antigen(s) of the cancer pulsed onto HLA-A2-expressing antigen-presenting cells. Alternatively, the T-cells can be re-stimulated with irradiated, autologous lymphocytes or with irradiated HLA-A2+ allogeneic lymphocytes and IL-2, for example.

The autologous T-cells can be modified to express a T-cell growth factor that promotes the growth and activation of the autologous T-cells. Suitable T-cell growth factors include, for example, interleukin (IL)-2, IL-7, IL-15, and IL-12. Suitable methods of modification are known in the art. See, for instance, Sambrook et al., MOLECULAR CLONING: A LABORATORY MANUAL, 3^{rd} ed., Cold Spring Harbor Press, Cold Spring Harbor, N.Y. 2001; and Ausubel et al., CURRENT PROTOCOLS IN MOLECULAR BIOLOGY, Greene Publishing Associates and John Wiley & Sons, N Y, 1994. In particular aspects, modified autologous T-cells express the T-cell growth factor at high levels. T-cell growth factor coding sequences, such as that of IL-12, are readily available in the art, as are promoters, the operable linkage of which to a T-cell growth factor coding sequence promote high-level expression.

2. Methods of Engineering of Host Cells

The immune cells (e.g., autologous or allogeneic T cells (e.g., memory T cells, Naïve T cells, regulatory T cells, CD4+ T cells, CD8+ T cells, or gamma-delta T cells), NK cells, invariant NK cells, or NKT cells can be genetically engineered to express antigen receptors such as engineered TCRs and/or chimeric antigen receptors (CARs). For example, the host cells (e.g., autologous or allogeneic T-cells) are modified to express a T cell receptor (TCR) having antigenic specificity for a cancer antigen. In particular embodiments, NK cells are engineered to express a TCR. The NK cells may be further engineered to express a CAR. Multiple CARs and/or TCRs, such as to different antigens, may be added to a single cell type, such as T cells or NK cells.

Suitable methods of modification are known in the art. See, for instance, Sambrook and Ausubel, supra. For example, the cells may be transduced to express a T cell receptor (TCR) having antigenic specificity for a cancer antigen using transduction techniques described in Heemskerk et al. (2008) and Johnson et al. (2009).

In some embodiments, the cells comprise one or more nucleic acids/expression constructs/vectors introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g., chimeric).

B. Engineered T Cells with Peptide Antigen Specificity

In one aspect of the disclosure, engineered target cells expressing a TCR having specificity for a peptide antigen. In embodiments of the disclosure, there are: a composition encoding α and β subunits of a TCR; and instructions for use of the composition. The composition may be, for example, a recombinant virus, or a viral vector. In some embodiments, the composition comprises one or more sequences encoding TCR subunits comprising at least one of: a variable region substantially as described herein.

In some embodiments, vectors can be used to introduce polynucleotide sequences that encode all or part of a functional TCR into a packaging cell line for the preparation of a recombinant virus. In addition to the elements as described herein, the vectors can contain polynucleotide sequences encoding the various components of the recombinant virus and at least one variable region as described herein, as well as any components necessary for the production of the virus that are not provided by the packaging cell line. In other embodiments, in addition to the elements as described herein, the vectors can contain polynucleotide sequences encoding the various components of the recombinant virus and at least one variable region as described here, as well as any components necessary for the production of the virus that are not provided by the packaging cell line. Eukaryotic cell expression vectors are well known in the art and are available from a number of commercial sources.

In some embodiments, one or more multicistronic expression vectors are utilized that include two or more of the elements (e.g., the viral genes, at least one of: an m1-α sequence and an m1-β sequence, a suicide gene or genes) necessary for production of a desired recombinant virus in packaging cells. The use of multicistronic vectors reduces the total number of vectors required and thus avoids the possible difficulties associated with coordinating expression from multiple vectors. In a multicistronic vector the various elements to be expressed are operably linked to one or more promoters (and other expression control elements as necessary). In some embodiments a multicistronic vector comprising a suicide gene and/or a reporter gene, viral elements and nucleotide sequences encoding all or part of an α or β subunit of a TCR, is used, wherein the nucleotide sequences are substantially as described herein.

Each component to be expressed in a multicistronic expression vector may be separated, for example, by an IRES element or a viral 2A element, to allow for separate expression of the various proteins from the same promoter. IRES elements and 2A elements are known in the art (U.S. Pat. No. 4,937,190; de Felipe et al., 2004. Traffic 5: 616-626, each of which is incorporated herein by reference in its entirety). In one embodiment, oligonucleotides encoding furin cleavage site sequences (RAKR) (Fang et al., 2005. Nat. Biotech 23: 584-590, which is incorporated herein by reference in its entirety) linked with 2A-like sequences from foot-and-mouth diseases virus (FMDV), equine rhinitis A virus (ERAV), and thosea asigna virus (TaV) (Szymczak et al., 2004. Nat. Biotechnol. 22: 589-594, which is incorporated herein by reference in its entirety) are used to separate genetic elements in a multicistronic vector. The efficacy of a particular multicistronic vector for use in synthesizing the desired recombinant virus can readily be tested by detecting expression of each of the genes using standard protocols. Exemplary protocols that are well known in the art include, but are not limited to, antibody-specific immunoassays such as Western blotting.

Vectors will usually contain a promoter that is recognized by the packaging cell and that is operably linked to the polynucleotide(s) encoding the targeting molecule, viral components, and the like. A promoter is an expression control element formed by a nucleic acid sequence that permits binding of RNA polymerase and transcription to occur. Promoters are untranslated sequences that are located upstream (5′) to the start codon of a structural gene (generally within about 100 to 1000 bp) and control the transcription and translation of the antigen-specific polynucleotide sequence to which they are operably linked. Promoters may be inducible or constitutive. The activity of the inducible promoters is induced by the presence or absence of biotic or abiotic factors. Inducible promoters can be a useful tool in genetic engineering because the expression of genes to which they are operably linked can be turned on or off at certain stages of development of an organism or in a particular tissue. Inducible promoters can be grouped as chemically-regulated promoters, and physically-regulated promoters. Typical chemically-regulated promoters include, not are not limited to, alcohol-regulated promoters (e.g., alcohol dehydrogenase I (alcA) gene promoter), tetracycline-regulated promoters (e.g., tetracycline-responsive promoter), steroid-regulated promoter (e.g., rat glucocorticoid receptor (GR)-based promoter, human estrogen receptor (ER)-based promoter, moth ecdysone receptor-based promoter, and the promoters based on the steroid/retinoid/thyroid receptor superfamily), metal-regulated promoters (e.g., metallothionein gene-based promoters), and pathogenesis-related promoters (e.g., Arabidopsis and maize pathogen-related (PR) protein-based promoters). Typical physically-regulated promoters include, but are not limited to, temperature-regulated promoters (e.g., heat shock promoters), and light-regulated promoters (e.g., soybean SSU promoter). Other exemplary promoters are described elsewhere, for example, in hypertext transfer protocol: world-wide-web at patentlens.net/daisy/promoters/768/271.html.

One of skill in the art will be able to select an appropriate promoter based on the specific circumstances. Many different promoters are well known in the art, as are methods for operably linking the promoter to the gene to be expressed. Both native promoter sequences and many heterologous promoters may be used to direct expression in the packaging cell and target cell. However, heterologous promoters are contemplated, as they generally permit greater transcription and higher yields of the desired protein as compared to the native promoter.

The promoter may be obtained, for example, from the genomes of viruses such as polyoma virus, fowlpox virus, adenovirus, bovine papilloma virus, avian sarcoma virus, cytomegalovirus, a retrovirus, hepatitis-B virus and Simian Virus 40 (SV40). The promoter may also be, for example, a heterologous mammalian promoter, e.g., the actin promoter or an immunoglobulin promoter, a heat-shock promoter, or the promoter normally associated with the native sequence, provided such promoters are compatible with the target cell. In one embodiment, the promoter is the naturally occurring viral promoter in a viral expression system.

Transcription may be increased by inserting an enhancer sequence into the vector(s). Enhancers are typically cis-acting elements of DNA, usually about 10 to 300 bp in length, that act on a promoter to increase its transcription. Many enhancer sequences are now known from mammalian genes (globin, elastase, albumin, α-fetoprotein, and insulin). An enhancer from a eukaryotic cell virus will be used is particularly contemplated. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the antigen-specific polynucleotide sequence and may be located at a site 5′ from the promoter.

Other vectors and methods suitable for adaptation to the expression of viral polypeptides, are well known in the art and are readily adapted to the specific circumstances.

Using the teachings provided herein, one of skill in the art will recognize that the efficacy of a particular expression system can be tested by transforming packaging cells with a vector comprising a gene encoding a reporter protein and measuring the expression using a suitable technique, for example, measuring fluorescence from a green fluorescent protein conjugate. Suitable reporter genes are well known in the art.

A vector that encodes a core virus is also known as a “viral vector.” There are a large number of available viral vectors that are suitable for use with the disclosure, including those identified for human gene therapy applications, such as those described by Pfeifer and Verma (Pfeifer, A. and I. M. Verma, 2001, Annu. Rev. Genomics Hum. Genet. 2:177-211, which is incorporated herein by reference in its entirety). Suitable viral vectors include vectors based on RNA viruses, such as retrovirus-derived vectors, e.g., Moloney murine leukemia virus (MLV)-derived vectors, and include more complex retrovirus-derived vectors, e.g., lentivirus-derived vectors. Human Immunodeficiency virus (HIV-1)-derived vectors belong to this category. Other examples include lentivirus vectors derived from HIV-2, feline immunodeficiency virus (Hy), equine infectious anemia virus, simian immunodeficiency virus (SIV) and maedi/visna virus.

The viral vector in particular may comprise one or more genes encoding components of the recombinant virus as well as nucleic acids encoding all or part of a functional MART-1 TCR. In some embodiments, the viral vector encodes components of the recombinant virus and at least one of: an m1-α variable region, an m1-β variable region and an m2-β variable region, and optionally, a suicide or reporter gene. In other embodiments, the viral vector encodes components of the recombinant virus and at least one of: an m1-α subunit, an m1-β subunit and an m2-β subunit, and optionally, a suicide or reporter gene. The viral vector may also comprise genetic elements that facilitate expression of the corresponding α and β polynucleotide sequences in a target cell, such as promoter and enhancer sequences. In order to prevent replication in the target cell, endogenous viral genes required for replication may be removed and provided separately in the packaging cell line.

In a particular embodiment the viral vector comprises an intact retroviral 5′ LTR and a self-inactivating 3′ LTR.

Any method known in the art may be used to produce infectious retroviral and/or lentiviral particles whose genome comprises an RNA copy of the viral vector. To this end, the viral vector (along with other vectors encoding at least one of: an m1-α subunit and an m1-β subunit of a TCR that recognizes a peptide antigen, and optionally, a suicide gene) may be introduced into a packaging cell line that packages viral genomic RNA based on the viral vector into viral particles.

The packaging cell line provides the viral proteins that are required in trans for the packaging of the viral genomic RNA into viral particles. The packaging cell line may be any cell line that is capable of expressing retroviral proteins. Particular packaging cell lines include 293 (ATCC CCL X), Platinum A, HeLa (ATCC CCL 2), D17 (ATCC CCL 183), MDCK (ATCC CCL 34), BHK (ATCC CCL-10) and Cf2Th (ATCC CRL 1430). The packaging cell line may stably express the necessary viral proteins. Such a packaging cell line is described, for example, in U.S. Pat. No. 6,218,181, which is incorporated herein by reference in its entirety. Alternatively a packaging cell line may be transiently transfected with plasmids comprising nucleic acid that encodes one or more necessary viral proteins, including, but not limited to, gag, pol, rev, and any envelope protein that facilitates transduction of a target cell, along with the viral vectors encoding at least one of an m1-α subunit and an m1-β subunit of a TCR that recognizes a peptide antigen.

Viral particles comprising a polynucleotide containing a gene of interest, which typically includes at least one of: an m1-α variable region nucleotide sequence, an m1-β variable region nucleotide sequence, an m2-β variable region nucleotide sequence, and optionally, a suicide or reporter gene, are collected and allowed to infect the target cell. In some embodiments, the gene of interest includes at least one of: an m1-α subunit nucleotide sequence, an m1-β subunit nucleotide sequence and an m2-β subunit nucleotide sequence. In some embodiments, the virus is pseudotyped to achieve target cell specificity. Methods for pseudotyping are well known in the art and also described herein.

In one embodiment, the recombinant virus used to deliver the gene of interest is a modified lentivirus and the viral vector is based on a lentivirus. As lentiviruses are able to infect both dividing and non-dividing cells, in this embodiment it is not necessary for target cells to be dividing (or to stimulate the target cells to divide).

In another embodiment, the recombinant virus used to deliver the gene of interest is a modified gammaretrovirus and the viral vector is based on a gammaretrovirus.

In another embodiment the vector is based on the murine stem cell virus (MSCV; (Hawley, R. G., et al. (1996) Proc. Natl. Acad. Sci. USA 93:10297-10302; Keller, G., et al. (1998) Blood 92:877-887; Hawley, R. G., et al. (1994) Gene Ther. 1:136-138, each of the foregoing which is incorporated herein by reference in its entirety). The MSCV vector provides long-term stable expression in target cells, particularly hematopoietic precursor cells and their differentiated progeny.

In another embodiment, the vector is based on a modified Moloney virus, for example a Moloney Murine Leukemia Virus. The viral vector can also can be based on a hybrid virus such as that described in Choi, J. K., et al. (2001. Stem Cells 19, No. 3, 236-246, which is incorporated herein by reference in its entirety).

A DNA viral vector may be used, including, for example adenovirus-based vectors and adeno-associated virus (AAV)-based vectors. Likewise, retroviral-adenoviral vectors also can be used with the methods of the disclosure.

Other vectors also can be used for polynucleotide delivery including vectors derived from herpes simplex viruses (HSVs), including amplicon vectors, replication-defective HSV and attenuated HSV (Krisky D M, Marconi P C, Oligino T J, Rouse R J, Fink D J, et al. 1998. Development of herpes simplex virus replication-defective multigene vectors for combination gene therapy applications. Gene Ther. 5: 1517-30, which is incorporated herein by reference in its entirety).

Other vectors that have recently been developed for gene therapy uses can also be used with the methods of the disclosure. Such vectors include those derived from baculoviruses and alpha-viruses. Jolly D J. 1999. Emerging viral vectors. pp 209-40 in Friedmann T, ed. 1999. The development of human gene therapy. New York: Cold Spring Harbor Lab, which is incorporated herein by reference in its entirety.

In some particular embodiments, the viral construct comprises sequences from a lentivirus genome, such as the HIV genome or the SIV genome. The viral construct may comprise sequences from the 5′ and 3′ LTRs of a lentivirus. More particularly, the viral construct comprises the R and U5 sequences from the 5′ LTR of a lentivirus and an inactivated or self-inactivating 3′ LTR from a lentivirus. The LTR sequences may be LTR sequences from any lentivirus from any species. For example, they may be LTR sequences from HIV, SIV, FIV or BIV. In particular, the LTR sequences are HIV LTR sequences.

The viral construct may comprise an inactivated or self-inactivating 3′ LTR. The 3′ LTR may be made self-inactivating by any method known in the art. In a particular embodiment the U3 element of the 3′ LTR contains a deletion of its enhancer sequence, such as the TATA box, Spl and NF-kappa B sites. As a result of the self-inactivating 3′ LTR, the provirus that is integrated into the host cell genome will comprise an inactivated 5′ LTR.

Optionally, the U3 sequence from the lentiviral 5′ LTR may be replaced with a promoter sequence in the viral construct. This may increase the titer of virus recovered from the packaging cell line. An enhancer sequence may also be included. Any enhancer/promoter combination that increases expression of the viral RNA genome in the packaging cell line may be used. In a particular embodiment the CMV enhancer/promoter sequence is used.

In some embodiments, the viral construct may comprise an inactivated or self-inactivating 3′ LTR. The 3′ LTR may be made self-inactivating by any method known in the art. In a particular embodiment, the U3 element of the 3′ LTR contains a deletion of its enhancer sequence, such as the TATA box, Spl and NF-kappa B sites. As a result of the self-inactivating 3′ LTR, the provirus that is integrated into the host cell genome will comprise an inactivated 5′ LTR.

The viral construct generally comprises a gene of interest, which typically includes at least one of: an m1-α variable region nucleotide sequence, an m1-β variable region nucleotide sequence, an m2-β variable region nucleotide sequence, an m1-α subunit nucleotide sequence, an m1-β subunit nucleotide sequence and an m2-β subunit nucleotide sequence, and optionally, a suicide or reporter gene that is desirably expressed in one or more target cells. The gene of interest may located between the 5′ LTR and 3′ LTR sequences. Further, the gene of interest may in particular be in a functional relationship with other genetic elements, for example transcription regulatory sequences such as promoters and/or enhancers, to regulate expression of the gene of interest in a particular manner once the gene is incorporated into the target cell. In certain embodiments, the useful transcriptional regulatory sequences are those that are highly regulated with respect to activity, both temporally and spatially.

In some embodiments, the gene of interest is in a functional relationship with internal promoter/enhancer regulatory sequences. An “internal” promoter/enhancer is one that is located between the 5′ LTR and the 3′ LTR sequences in the viral construct and is operably linked to the gene that is desirably expressed.

The internal promoter/enhancer may be any promoter, enhancer or promoter/enhancer combination known to increase expression of a gene with which it is in a functional relationship. A “functional relationship” and “operably linked” mean, without limitation, that the gene is in the correct location and orientation with respect to the promoter and/or enhancer that expression of the gene will be affected when the promoter and/or enhancer is contacted with the appropriate molecules.

The internal promoter/enhancer may be selected based on the desired expression pattern of the gene of interest and the specific properties of known promoters/enhancers. Thus, the internal promoter may be a constitutive promoter. Non-limiting examples of constitutive promoters that may be used include the promoter for ubiquitin, CMV (Karasuyama et al., 1989. J. Exp. Med. 169:13, which is incorporated herein by reference in its entirety), beta-actin (Gunning et al., 1989. Proc. Natl. Acad. Sci. USA 84:4831-4835, which is incorporated herein by reference in its entirety) and pgk (see, for example, Adra et al., 1987. Gene 60:65-74; Singer-Sam et al., 1984. Gene 32:409-417; and Dobson et al., 1982. Nucleic Acids Res. 10:2635-2637, each of the foregoing which is incorporated herein by reference in its entirety).

In addition, promoters may be selected to allow for inducible expression of the gene. A number of systems for inducible expression are known in the art, including the tetracycline responsive system and the lac operator-repressor system. It is also contemplated that a combination of promoters may be used to obtain the desired expression of the gene of interest. The skilled artisan will be able to select a promoter based on the desired expression pattern of the gene in the organism and/or the target cell of interest.

C. Chimeric Antigen Receptors

“Chimeric antigen receptors” (CARs), as used herein, refer to engineered receptors that are capable of grafting a desired specificity to an antigen into an immune effector cells, such as T cells and NK cells. Typically, a CAR protein comprises an extracellular domain that introduces the desired specificity, a transmembrane domain and an intracellular domain that transmits a signal to the immune effector cells when the immune effector cells bind to the antigen. In certain embodiments, the extracellular domain comprises a leader peptide, an antigen recognition region and a spacer region. In certain embodiments, the antigen recognition region is derived from an antibody that specifically binds to the antigen. In certain embodiments, the antigen recognition region is a single—chain variable fragment (scFv) derived from the antibody. In certain embodiment, the single-chain variable fragment comprises a heavy chain variable region fused to a light chain variable region through a flexible linker.

The term “leader peptide” as referred to herein is used according to its ordinary meaning in the art and refers to a peptide having a length of about 5-30 amino acids. A leader peptide is present at the N-terminus of newly synthesized proteins that form part of the secretory pathway. Proteins of the secretory pathway include but are not limited to proteins that reside either inside certain organelles (the endoplasmic reticulum, Golgi or endosomes), are secreted from the cell, or are inserted into a cellular membrane. In some embodiments, the leader peptide forms part of the transmembrane domain of a protein.

In one aspect, the present disclosure provides a CAR protein that binds to an antigen described herein. In some embodiments, the CAR protein includes from the N-terminus to the C-terminus: a leader peptide, an anti-antigen heavy chain variable domain, a linker domain, an anti-antigen light chain variable domain, a CD8α hinge region, a CD8α transmembrane domain (or a CD28 transmembrane domain), a 4-1BB intracellular co-stimulatory signaling domain (or a CD28 intracellular co-stimulatory signaling domain, or a CD28 intracellular co-stimulatory signaling domain followed by a 4-1BB intracellular co-stimulatory signaling domain) and a CD3-ζ intracellular T cell signaling domain.

In some embodiments, the protein includes from the N-terminus to the C-terminus: a CD8α leader peptide, an antigen HuCAR scFV, a human CD8α hinge domain, a CD28 transmembrane domain, the zeta (c) chain of the human CD3 complex T-cell signaling domain.

In other embodiments, the protein includes from the N-terminus to the C-terminus: a CD8α leader peptide, an antigen HuCAR scFV, a human CD8α hinge domain, a 4-1BB intracellular co-stimulatory signaling domain, and the zeta (c) chain of the human CD3 complex T-cell signaling domain.

In an alternative embodiment, the protein includes from the N-terminus to the C-terminus: a CD8α leader peptide, an antigen HuCAR scFV, a human CD8α hinge domain, a 4-1BB intracellular co-stimulatory signaling domain, a CD28 transmembrane domain, and the zeta (c) chain of the human CD3 complex T-cell signaling domain.

In another embodiment, the protein includes from the N-terminus to the C-terminus: a leader peptide, an antigen heavy chain variable domain, a linker domain, an antigen light chain variable domain, a human IgG1-CH2-CH3 domain, a spacer region, a CD28 transmembrane domain, a 4-1BB intracellular co-stimulatory signaling and the zeta (c) chain of the human CD3 complex T-cell signaling domain.

In some embodiments, the nucleic acid encodes the antibody heavy chain variable domain and the antibody light chain variable domain from an antibody that binds the antigen.

In another aspect, an expression vector including a nucleic acid provided herein including embodiments thereof is provided. In another aspect, a T lymphocyte including the expression vector provided herein including embodiments thereof is provided. In another aspect, a mammalian cell including the expression vector provided herein including embodiments thereof is provided. In another aspect, a recombinant protein is provided. The recombinant protein includes (i) an antibody region including a central cavity formed by a heavy chain variable (VH) region and a light chain variable (VL) region, wherein the central cavity forms a peptide binding site including framework region amino acid residues; and (ii) a transmembrane domain.

In another aspect, a recombinant protein is provided. The recombinant protein includes a first portion including an antibody heavy chain variable domain and a second portion including an antibody light chain variable domain and an antibody light chain constant domain, wherein the first portion further includes a transmembrane domain, and wherein the antibody heavy chain variable domain, the antibody light chain variable domain and the antibody light chain constant domain together form an antibody region.

In another aspect, a recombinant protein is provided. The recombinant protein includes a first portion including an antibody heavy chain variable domain and a second portion including an antibody light chain variable domain, wherein the first portion further includes a transmembrane domain, and wherein the antibody heavy chain variable domain and the antibody light chain variable domain together form an antibody region.

In another aspect, a recombinant protein is provided. The recombinant protein includes a first portion including an antibody heavy chain variable domain and an antibody heavy chain constant domain, and a second portion including an antibody light chain variable domain, wherein the first portion further includes a transmembrane domain, and wherein the antibody heavy chain variable domain, the antibody heavy chain constant domain and the antibody light chain variable domain together form an antibody region.

In another aspect, a recombinant protein is provided. The recombinant protein includes a first portion including an antibody heavy chain variable domain and a second portion including an antibody light chain variable domain and an antibody light chain constant domain, wherein the second portion further includes a transmembrane domain, and wherein the antibody heavy chain variable domain, the antibody light chain variable domain and the antibody light chain constant domain together form an antibody region.

In another aspect, a recombinant protein is provided. The recombinant protein includes a first portion including an antibody heavy chain variable domain and a second portion including an antibody light chain variable domain, wherein the second portion further includes a transmembrane domain, and wherein the antibody heavy chain variable domain and the antibody light chain variable domain together form an antibody region.

In another aspect, a mammalian cell including the recombinant protein provided herein including embodiments thereof is provided, wherein the transmembrane domain is within the cell membrane of the mammalian cell.

In some embodiments, the transmembrane domain is a CD8α transmembrane domain. The term “CD8α transmembrane domain” as provided herein includes any of the recombinant or naturally-occurring forms of the transmembrane domain of CD8a. In some aspects, the variants or homologs have at least 90%, 95%, 96%, 97%, 98%, 99% or 100% amino acid sequence identity across the whole sequence or a portion of the sequence compared to a naturally occurring CD8α transmembrane domain polypeptide. In some embodiments, the CD8α transmembrane domain has the polypeptide sequence of IYIWAPLAGTCGVLLLSLVIT (SEQ ID NO: 1). In some embodiments, the CD8α transmembrane domain is the protein encoded by the nucleic acid sequence of

(SEQ ID NO: 2)
ATCTACATCTGGGCTCCACTGGCAGGAACCTGTGGCGTGCTGCTGCTGT
CCCTGGTCATCACA.

In some embodiments, the transmembrane domain is a CD28 transmembrane domain. The term “CD28 transmembrane domain” as provided herein includes any of the recombinant or naturally-occurring forms of the transmembrane domain of CD28, or variants or homologs thereof that maintain CD28 transmembrane domain activity. In some aspects, the variants or homologs have at least 90%, 95%, 96%, 97%, 98%, 99% or 100% amino acid sequence identity across the whole sequence or a portion of the sequence compared to a naturally occurring CD28 transmembrane domain polypeptide. In some embodiments, the CD28 transmembrane domain has the polypeptide sequence of FWVLVVVGGVLACYSLLVTVAFIIFWV (SEQ ID NO: 3). In some embodiments, the CD28 transmembrane domain is the protein encoded by the nucleic acid sequence of

(SEQ ID NO: 4)
TTTTGGGTGCTGGTGGTGGTTGGTGGAGTCCTGGCTTGCTATAGCTTGC
TAGTAACAGTGGCCTTTATTATTTTCTGGGTG.

In some embodiments, the intracellular T cell signaling domain is a CD3-ζ intracellular T cell signaling domain. In some embodiments, the intracellular T cell signaling domain includes the signaling domain of the zeta (ζ) chain of the human CD3 complex. In some embodiments, the intracellular T cell signaling domain is a CD3-ζ intracellular T cell signaling domain. In some embodiments, the intracellular T cell signaling domain is the protein encoded by the nucleic acid sequence of

(SEQ ID NO: 5)
AGAGTGAAGTTCAGCAGGAGCGCAGACGCCCCCGCGTACCAGCAGGGCC
AGAACCAGCTCTATAACGAGCTCAATCTAGGACGAAGAGAGGAGTACGA
TGTTTTGGACAAGAGACGTGGCCGGGACCCTGAGATGGGGGGAAAGCCG
AGAAGGAAGAACCCTCAGGAAGGCCTGTACAATGAACTGCAGAAAGATA
AGATGGCGGAGGCCTACAGTGAGATTGGGATGAAAGGCGAGCGCCGGAG
GGGCAAGGGGCACGATGGCCTTTACCAGGGTCTCAGTACAGCCACCAAG
GACACCTACGACGCCCTTCACATGCAGGCCCTGCCCCCTCGCTAA.

In some embodiments, the isolated nucleic acid provided herein includes an intracellular co-stimulatory signaling sequence encoding an intracellular co-stimulatory signaling domain. An “intracellular co-stimulatory signaling domain” as provided herein includes amino acid sequences capable of providing co-stimulatory signaling in response to binding of an antigen to the antibody region provided herein including embodiments thereof. In some embodiments, the signaling of the co-stimulatory signaling domain results in the production of cytokines and proliferation of the T cell expressing the same. In some embodiments, the intracellular co-stimulatory signaling domain is a CD28 intracellular co-stimulatory signaling domain, a 4-1BB intracellular co-stimulatory signaling domain. In some embodiments, the intracellular co-stimulatory signaling domain includes a CD28 intracellular co-stimulatory signaling domain, a 4-1BB intracellular co-stimulatory signaling domain, an ICOS intracellular co-stimulatory signaling domain, an OX-40 intracellular co-stimulatory signaling domain or any combination thereof. In some embodiments, the CD28 co-stimulating domain has the polypeptide sequence of RSKRSRLLHSDYMNMTPRRPGPTRKHYQPYAPPRDFAAYRS (SEQ ID NO: 6). In some embodiments, the CD28 intracellular co-stimulatory signaling domain is the protein encoded by the nucleic acid sequence of AGGAGTAAGAGGAGCAGGCTCCTGCACAGTGACTACATGAACATGACTCCCCGC CGCCCCGGGCCCACCCGCAAGCATTACCAGCCCTATGCCCCACCACGCGACTTCG CAGCCTATCGCTCC (SEQ ID NO: 7). In some embodiments, the 4-1BB intracellular co-stimulatory signaling domain has the polypeptide sequence of KRGRKKLLYIFKQPFMRPVQTTQEEDGCSCRFPEEEEGGCEL (SEQ ID NO: 8). In some embodiments, the 4-1BB intracellular co-stimulatory signaling domain is the protein encoded by the nucleic acid sequence of

(SEQ ID NO: 9)
AAACGGGGCAGAAAGAAACTCCTGTATATATTCAAACAACCATTTATGA
GACCAGTACAAACTACTCAAGAGGAAGATGGCTGTAGCTGCCGATTTCC
AGAAGAAGAAGAAGGAGGATGTGAACTG.

In some embodiments, the isolated nucleic acid provided herein includes a spacer sequence encoding a spacer region. A “spacer region” as provided herein is a polypeptide connecting the antibody region with the transmembrane domain, or connecting various components of the antibody region. In some embodiments, the spacer region is between the antibody region and the transmembrane domain. In some embodiments, the spacer region connects the heavy chain variable region with the transmembrane domain. In some embodiments, the spacer region connects the heavy chain constant region with the transmembrane domain. In some embodiments, the spacer region connects the light chain variable region with the transmembrane domain. In some embodiments, the spacer region connects the light chain constant region with the transmembrane domain. In some embodiments, the binding affinity of the antibody region to an antigen is increased compared to the absence of the spacer region. In some embodiments, the steric hindrance between an antibody region and an antigen is decreased in the presence of the spacer region.

In some embodiments, the spacer region includes a hinge region. In some embodiments, the hinge region is a CD8α hinge region. In some embodiments, the hinge region is a CD28 hinge region.

In some embodiments, the spacer region includes a Fc region. Examples of spacer regions contemplated for the compositions and methods provided herein include without limitation, immunoglobulin molecules or fragments thereof (e.g., IgG1, IgG2, IgG3, IgG4) and immunoglobulin molecules or fragments thereof (e.g., IgG1, IgG2, IgG3, IgG4) including mutations affecting Fc receptor binding. In some embodiments, the spacer region is a fragment of an IgG (e.g., IgG4), wherein the fragment includes a deletion of the CH2 domain. The spacer region may be a peptide linker. In some embodiments, the nucleic acid does not include a spacer sequence encoding a spacer region.

In some embodiments, the spacer region connects various components of the antibody region. In some embodiments, the spacer region connects the heavy chain variable region with the light chain variable region.

In some embodiments, the isolated nucleic acid provided herein includes a linker sequence encoding a linker domain. In some embodiment, the linker domain is inserted between the VH and VL of the scFv. In some embodiments, the linker domain is between the transmembrane domain and the intracellular T cell signaling domain. In some embodiments, the linker domain is between the intracellular T cell signaling domain and the intracellular co-stimulatory signaling domain. In some embodiments, the linker domain comprises the sequence GGGGSGGGGSGGGGS (SEQ ID NO: 10).

In some embodiments, the isolated nucleic acid provided herein does not include a linker sequence encoding a linker domain.

In some embodiments, the nucleic acid includes (i) a heavy chain sequence encoding a heavy chain domain of the protein, the heavy chain domain includes a variable heavy chain domain and the transmembrane domain; and (ii) a light chain sequence encoding a light chain domain of the protein, the light chain domain includes a variable light chain domain, wherein the variable heavy chain domain and the variable light chain domain together form at least a portion of the antibody region.

In some embodiments, the nucleic acid includes (i) a heavy chain sequence encoding a heavy chain domain of the protein, the heavy chain domain includes a variable heavy chain domain; and (ii) a light chain sequence encoding a light chain domain of the protein, the light chain domain includes a variable light chain domain and a transmembrane domain, wherein the variable heavy chain domain and the variable light chain domain together form at least a portion of the antibody region.

A “heavy chain sequence” as provided herein refers to the nucleic acid sequence encoding for a heavy chain domain provided herein. A heavy chain domain provided herein may include heavy chain variable (VH) region and/or a heavy chain constant region (CH). A “light chain sequence” as provided herein refers to the nucleic acid sequence encoding for a light chain domain provided herein. A light chain domain provided herein may include a light chain variable (VL) region and/or a light chain constant region (CL). The term “heavy chain domain” as referred to herein is used according to its ordinary meaning in the art and refers to a polypeptide including a heavy chain variable (VH) region and a heavy chain constant region (CH). The term “light chain domain” as referred to herein is used according to its ordinary meaning in the art and refers to a polypeptide including a light chain variable (VL) region and a light chain constant region (CL). In some embodiments, the antibody heavy chain variable domain and the antibody light chain variable domain are humanized.

In some embodiments, the protein or antibody region provided herein including embodiments thereof competes for antigen binding with, specifically binds to the same antigen or epitope as, and/or contains one, more, or all CDRs (or CDRs comprising at least at or about 75, 80, 85, 90, 91, 92, 93, 94, 95, 96, 97, 98, or 99% identity to the CDRs), e.g., including a heavy chain CDR 1, 2, and/or 3 and/or a light chain CDR1, 2, and/or 3, of antibody that binds the antigen.

In some embodiments, the nucleic acid encodes the antibody heavy chain variable domain and the antibody light chain variable domain from an antibody that binds the antigen.

In some embodiments, the protein includes an intracellular co-stimulatory signaling domain and a CD3-ζ intracellular T cell signaling domain. In some embodiments, the protein includes from the amino terminus to the carboxyl terminus: a heavy chain variable domain, a light chain variable domain, a transmembrane domain, an intracellular co-stimulatory signaling domain and a CD3-ζ intracellular T cell signaling domain.

In some embodiments, the protein includes an intracellular co-stimulatory signaling domain and a CD3-ζ intracellular T cell signaling domain. In some embodiments, the protein includes from the amino terminus to the carboxyl terminus: a light chain variable domain, a heavy chain variable domain, a transmembrane domain, an intracellular co-stimulatory signaling domain and a CD3-ζ intracellular T cell signaling domain.

In some embodiments, the recombinant protein includes a first portion including an antibody heavy chain variable domain and a second portion including an antibody light chain variable domain. In some embodiments, the first portion includes an intracellular co-stimulatory signaling domain and a CD3-ζ intracellular T cell signaling domain. In some embodiments, the first portion includes from the amino terminus to the carboxyl terminus: a heavy chain variable domain, a transmembrane domain, an intracellular co-stimulatory signaling domain and a CD3-ζ intracellular T cell signaling domain.

In some embodiments, the recombinant protein includes a first portion including an antibody heavy chain variable domain and a heavy chain constant domain, and a second portion including an antibody light chain variable domain. In some embodiments, the first portion includes an intracellular co-stimulatory signaling domain and a CD3-ζ intracellular T cell signaling domain. In some embodiments, the first portion includes from the amino terminus to the carboxyl terminus: the heavy chain variable domain, a heavy chain constant domain, a transmembrane domain, an intracellular co-stimulatory signaling domain and a CD3-ζ intracellular T cell signaling domain.

In some embodiments, the protein includes a CD3-ζ intracellular T cell signaling domain and intracellular co-stimulatory signaling domain. In some embodiments, the protein includes from the amino terminus to the carboxyl terminus: a heavy chain variable domain, a light chain variable domain, a transmembrane domain, a CD3-ζ intracellular T cell signaling domain and an intracellular co-stimulatory signaling domain.

In some embodiments, the recombinant protein includes a first portion including an antibody heavy chain variable domain and a second portion including an antibody light chain variable domain. In some embodiments, the first portion includes a CD3-ζ intracellular T cell signaling domain and intracellular co-stimulatory signaling domain. In some embodiments, the first portion includes from the amino terminus to the carboxyl terminus: a heavy chain variable domain, a transmembrane domain, a CD3-ζ intracellular T cell signaling domain and an intracellular co-stimulatory signaling domain.

In some embodiments, the recombinant protein includes a first portion including an antibody heavy chain variable domain and a heavy chain constant domain, and a second portion including an antibody light chain variable domain. In some embodiments, the first portion includes a CD3-ζ intracellular T cell signaling domain and intracellular co-stimulatory signaling domain. In some embodiments, the first portion includes from the amino terminus to the carboxyl terminus: a heavy chain variable domain, a heavy chain constant domain, a transmembrane domain, a CD3-ζ intracellular T cell signaling domain and an intracellular co-stimulatory signaling domain.

In some embodiments, the isolated nucleic acid encodes a protein from the N-terminus to the C-terminus: a leader peptide, an anti-antigen heavy chain variable domain, a linker domain, an anti-antigen light chain variable domain, a human IgG1-CH2-CH3 domain, a spacer region, a CD28 domain, a 4-1BB intracellular co-stimulatory signaling domain and a CD3-ζ intracellular T cell signaling domain.

In some embodiments, the isolated nucleic acid encodes a protein from the N-terminus to the C-terminus: a leader peptide, an anti-antigen heavy chain variable domain, a linker domain, an anti-antigen light chain variable domain, a spacer region, a CD28 domain, a 4-1BB intracellular co-stimulatory signaling domain and a CD3-ζ intracellular T cell signaling domain.

In some embodiments, the isolated nucleic acid encodes a protein from the N-terminus to the C-terminus: a leader peptide, an anti-antigen heavy chain variable domain, a linker domain, an anti-antigen light chain variable domain, a spacer region, a CD28 transmembrane and co-stimulatory domain, and a CD3-ζ intracellular T cell signaling domain.

In some embodiments, the isolated nucleic acid encodes a protein from the N-terminus to the C-terminus: a leader peptide, an anti-antigen heavy chain variable domain, a linker domain, an anti-antigen light chain variable domain, a spacer region, a CD8α transmembrane domain (or a CD28 transmembrane domain), a 4-1BB intracellular co-stimulatory signaling domain and a CD3-ζ intracellular T cell signaling domain.

In certain embodiments, the antigen CAR protein provided herein demonstrates a high affinity to antigen. In certain embodiments, the CAR protein provided herein has a binding affinity to antigen (EC₅₀ as measured by ELISA) of less than 1 nM, 0.9 nM, 0.8 nM, 0.7 nM, 0.6 nM, 0.5 nM, 0.4 nM, 0.3 nM, 0.2 nM, 0.1 nM, 0.09 nM, 0.08 nM, 0.07 nM, 0.06 nM or 0.05 nM. For the purposes of this application, ELISA EC₅₀ values may be determined as follows. antigen-4 extracellular domain protein (with 6 HIS tag at the C-terminus) was produced recombinantly in HEK293 cells and coated onto a high binding 96-well clear plate (Corning-Costar, Fisher Scientific) at 1 μg/ml concentration (100 μl/well) at 4° C. for 14 to 16 hours. The coated plates were washed with PBS, pH 7.4, briefly and blocked with 200 μl/well of 5% non-fat milk in PBS for 2 hours at 37° C. Serial dilutions of the testing monoclonal antibodies (IgGs or scFvs fragments), starting from 10 μg/ml and 3-fold titration down for 12 steps, were added to the 96-well plate for binding by incubating 45 minutes at 37° C. with a cover on the assay plate. Then the plates were washed with PBS containing Tween 20 (0.05% concentration) for 3 times and PBS one time. Secondary antibody of anti-human or anti-rabbit, or other species IgG specific antibodies with HRP conjugate (Jackson ImmunoResearch) was added for incubation at room temperature for 1 hour per manufacturer's suggested dilution. Detection was conducted by adding HRP substrate, TMB (ThermoFisher) for 10 minutes, and stopped by adding 50 μl/well of 2N H₂SO₄. The plates were read for absorbance at 450 nm using a plate reader (SpectraMax M4, Molecular Devices). Data were collected and graphed using a 4-parameter fitting curve with GrapPad Prism 7 software for EC₅₀ calculation.

In another aspect, a T lymphocyte including the recombinant protein provided herein including embodiments thereof is provided, wherein the transmembrane domain is within the cell membrane of the T lymphocyte.

D. Vaccines

Cancer vaccines are a form of active immunotherapy where cell composition or “vaccine” is administered to a subject. Vaccines may be administered systemically, such as intranvenously or intradermally. Vaccines may also be administered multiple times to enhance the immune response against the administered antigens.

1. Adjuvants

As also well known in the art, the immunogenicity of a particular immunogen composition can be enhanced by the use of non-specific stimulators of the immune response, known as adjuvants. Adjuvants have been used experimentally to promote a generalized increase in immunity against poorly immunogenic antigens (e.g., U.S. Pat. No. 4,877,611). Immunization protocols have used adjuvants to stimulate responses for many years, and as such adjuvants are well known to one of ordinary skill in the art. Some adjuvants affect the way in which antigens are presented. For example, the immune response is increased when protein antigens are adsorbed to alum. Emulsification of antigens also prolongs the duration of antigen presentation and initiates an innate immune response. Suitable molecule adjuvants include all acceptable immunostimulatory compounds, such as cytokines, toxins or synthetic compositions.

In some aspects, the compositions described herein may further comprise another adjuvant. Although Alum is an approved adjuvant for humans, adjuvants in experimental animals include complete Freund's adjuvant (a non-specific stimulator of the immune response containing killed Mycobacterium tuberculosis), incomplete Freund's adjuvants and aluminum hydroxide adjuvant. Other adjuvants that may also be used in animals and sometimes humans include Interleukin (IL)-1, IL-2, IL-4, IL-7, IL-12, interferon, Bacillus Calmeit-Guérin (BCG), aluminum hydroxide, muramyl dipeptide (MDP) compounds, such as thur-MDP and nor-MDP (N-acetylmuramyl-L-alanyl-D-isoglutamine MDP), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion also is contemplated. MHC antigens may even be used.

In one aspect, and approved for humans, an adjuvant effect is achieved by use of an agent, such as alum, used in about 0.05 to about 0.1% solution in phosphate buffered saline. Alternatively, in experimental animals the antigen is made as an admixture with synthetic polymers of sugars (Carbopol®) used as an about 0.25% solution. Adjuvant effects may also be achieved by aggregation of the antigen in the vaccine by heat treatment with temperatures ranging between about 70° to about 101° C. for a 30 second to 2-minute period, respectively. Aggregation by reactivating with pepsin treated (Fab) antibodies to albumin, mixture with bacterial cell(s) such as C. parvum, an endotoxin or a lipopolysaccharide component of Gram-negative bacteria, emulsion in physiologically acceptable oil vehicles, such as mannide mono-oleate (Aracel A), or emulsion with a 20% solution of a perfluorocarbon (Fluosol-DA®) used as a block substitute, also may be employed.

Some adjuvants, for example, certain organic molecules obtained from bacteria, act on the host rather than on the antigen. An example is MDP, a bacterial peptidoglycan. The effects of MDP, as with most adjuvants, are not fully understood, although researchers are now beginning to understand that they activate cells of the innate immune system, e.g. dendritic cells, macrophages, neutrophils, NKT cells, NK cells, etc. MDP stimulates macrophages but also appears to stimulate B cells directly. The effects of adjuvants, therefore, are not antigen-specific. If they are administered together with a purified antigen, however, they can be used to selectively promote the response to the antigen.

In certain embodiments, hemocyanins and hemoerythrins may also be used in the compositions of the present disclosure. The use of hemocyanin from keyhole limpet (KLH) is used in certain embodiments, although other molluscan and arthropod hemocyanins and hemoerythrins may be employed.

Various polysaccharide adjuvants may also be used. For example, the use of various pneumococcal polysaccharide adjuvants on the antibody responses of mice has been described (Yin et al., 1989). The doses that produce optimal responses, or that otherwise do not produce suppression, should be employed as indicated (Yin et al., 1989). Polyamine varieties of polysaccharides are particularly contemplated, such as chitin and chitosan, including deacetylated chitin.

Another group of adjuvants are the muramyl dipeptide (MDP, N-acetylmuramyl-L-alanyl-D-isoglutamine) group of bacterial peptidoglycans. Derivatives of muramyl dipeptide, such as the amino acid derivative threonyl-MDP, and the fatty acid derivative muramyl tripeptide phosphatidylethanolamide (MTPPE) are also contemplated.

U.S. Pat. No. 4,950,645 describes a lipophilic disaccharide-tripeptide derivative of muramyl dipeptide which is described for use in artificial liposomes formed from phosphatidyl choline and phosphatidyl glycerol. This is effective in activating human monocytes and destroying tumor cells but is non-toxic in generally high doses. The compounds of U.S. Pat. No. 4,950,645 and PCT Patent Application WO 91/16347, are contemplated for use with cellular carriers and other embodiments of the present disclosure.

BCG and BCG-cell wall skeleton (CWS) may also be used as adjuvants, with or without trehalose dimycolate. Trehalose dimycolate may be used itself. Trehalose dimycolate administration has been shown to correlate with augmented resistance to influenza virus infection in mice (Azuma et al., 1988). Trehalose dimycolate may be prepared as described in U.S. Pat. No. 4,579,945. BCG is an important clinical tool because of its immunostimulatory properties. BCG acts to stimulate the reticuloendothelial system (RES), activates natural killer (NK) cells and increases proliferation of hematopoietic stem cells. Cell wall extracts of BCG have proven to have excellent immune adjuvant activity. Molecular genetic tools and methods for mycobacteria have provided the means to introduce foreign genes into BCG (Jacobs et al., 1987; Snapper et al., 1988; Husson et al., 1990; Martin et al., 1990). Live BCG is an effective and safe vaccine used worldwide to prevent tuberculosis. BCG and other mycobacteria are highly effective adjuvants, and the immune response to mycobacteria has been studied extensively. With nearly 2 billion immunizations, BCG has a long record of safe use in man (Luelmo, 1982; Lotte et al., 1984). It is one of the few vaccines that can be given at birth, it engenders long-lived immune responses with only a single dose, and there is a worldwide distribution network with experience in BCG vaccination. An exemplary BCG vaccine is sold as TICE BCG (Organon Inc., West Orange, N.J.).

Amphipathic and surface-active agents, e.g., saponin and derivatives such as QS21 (Cambridge Biotech), form yet another group of adjuvants for use with the immunogens of the present disclosure. Nonionic block copolymer surfactants (Rabinovich et al., 1994) may also be employed. Oligonucleotides are another useful group of adjuvants (Yamamoto et al., 1988). Quil A and lentinen are other adjuvants that may be used in certain embodiments of the present disclosure.

Another group of adjuvants are the detoxified endotoxins, such as the refined detoxified endotoxin of U.S. Pat. No. 4,866,034. These refined detoxified endotoxins are effective in producing adjuvant responses in mammals. Of course, the detoxified endotoxins may be combined with other adjuvants to prepare multi-adjuvant-incorporated cells. For example, combination of detoxified endotoxins with trehalose dimycolate is particularly contemplated, as described in U.S. Pat. No. 4,435,386. Combinations of detoxified endotoxins with trehalose dimycolate and endotoxic glycolipids is also contemplated (U.S. Pat. No. 4,505,899), as is combination of detoxified endotoxins with cCWS or CWS and trehalose dimycolate, as described in U.S. Pat. Nos. 4,436,727, 4,436,728 and 4,505,900. Combinations of just CWS and trehalose dimycolate, without detoxified endotoxins, are also envisioned to be useful, as described in U.S. Pat. No. 4,520,019.

Those of skill in the art will know the different kinds of adjuvants that can be conjugated to vaccines in accordance with this disclosure and which are approved for human vs experimental use. These include alkyl lysophosphilipids (ALP); BCG; and biotin (including biotinylated derivatives) among others. Certain adjuvants particularly contemplated for use are the teichoic acids from Gram⁻ bacterial cells. These include the lipoteichoic acids (LTA), ribitol teichoic acids (RTA) and glycerol teichoic acid (GTA). Active forms of their synthetic counterparts may also be employed in connection with the compositions of this disclosur (Takada et al., 1995).

Various adjuvants, even those that are not commonly used in humans, may still be employed in animals. Adjuvants may be encoded by a nucleic acid (e.g., DNA or RNA). It is contemplated that such adjuvants may be also be encoded in a nucleic acid (e.g., an expression vector) encoding the antigen, or in a separate vector or other construct. Nucleic acids encoding the adjuvants can be delivered directly, such as for example with lipids or liposomes.

2. Biological Response Modifiers (BRM)

In addition to adjuvants, it may be desirable to co-administer BRM, which have been shown to upregulate T cell immunity or downregulate suppressor cell activity. Such BRMs include, but are not limited to, cimetidine (CIM; 1200 mg/d) (Smith/Kline, Pa.); low-dose cyclophosphamide (CYP; 300 mg/m²) (Johnson/Mead, N.J.), cytokines such as interferon, IL-2, or IL-12 or genes encoding proteins involved in immune helper functions, such as B-7. Additional biological response modifiers include those described in Gupta and Kanodia (2002) and Bisht et al. (2010), both of which are incorporated herein by reference.

3. Chemokines

Chemokines, nucleic acids that encode for chemokines, and/or cells that express such also may be used as vaccine components. Chemokines generally act as chemoattractants to recruit immune effector cells to the site of chemokine expression. It may be advantageous to express a particular chemokine coding sequence in combination with, for example, a cytokine coding sequence, to enhance the recruitment of other immune system components to the site of treatment. Such chemokines include, for example, RANTES, MCAF, MIP1-α, MIP1-0, IP-10 and combinations thereof. The skilled artisan will recognize that certain cytokines are also known to have chemoattractant effects and could also be classified under the term chemokines.

### IV. METHODS OF USE

A. Treatments

In some embodiments, the present disclosure provides methods for immunotherapy comprising administering an effective amount of the immune cells of the present disclosure. In one embodiment, a medical disease or disorder is treated by transfer of an immune cell population that elicits an immune response. In certain embodiments of the present disclosure, cancer is treated by transfer of an immune cell population that elicits an immune response. Provided herein are methods for treating or delaying progression of cancer in an individual comprising administering to the individual an effective amount an antigen-specific cell therapy. The present methods may be applied for the treatment of immune disorders, solid cancers, or hematologic cancers.

In certain embodiments of the present disclosure, immune cells are delivered to an individual in need thereof, such as an individual that has cancer. The cells then enhance the individual's immune system to attack the respective cancer cells. In some cases, the individual is provided with one or more doses of the immune cells. In cases where the individual is provided with two or more doses of the immune cells, the duration between the administrations should be sufficient to allow time for propagation in the individual, and in specific embodiments the duration between doses is 1, 2, 3, 4, 5, 6, 7, or more days.

In certain embodiments, a growth factor that promotes the growth and activation of the immune cells is administered to the subject either concomitantly with the immune cells or subsequently to the immune cells. The immune cell growth factor can be any suitable growth factor that promotes the growth and activation of the immune cells. Examples of suitable immune cell growth factors include interleukin (IL)-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2.

Therapeutically effective amounts of immune cells can be administered by a number of routes, including parenteral administration, for example, intravenous, intraperitoneal, intramuscular, intrasternal, or intraarticular injection, or infusion.

The immune cell population can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective number of immune cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ immune cells/m². In a certain embodiment, the dose used in the treatment of human subjects ranges from about 3.8×10⁹ to about 3.8×10¹⁰ immune cells/m². In additional embodiments, a therapeutically effective number of immune cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight. The exact number of immune cells is readily determined by one of skill in the art based on the age, weight, sex, and physiological condition of the subject. Effective doses can be extrapolated from dose-response curves derived from in vitro or animal model test systems.

B. Pharmaceutical Compositions

Also provided herein are pharmaceutical compositions and formulations comprising immune cells (e.g., T cells, CAR-T cells, dendritic cells or NK cells) and a pharmaceutically acceptable carrier.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn— protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Patent Publication Nos. 2005/0260186 and 2006/0104968. In one aspect, a sHASEGP is combined with one or more additional glycosaminoglycanases such as chondroitinases.

C. Combination Therapies

In certain embodiments, the compositions and methods of the present embodiments involve an immune cell population in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

An immune cell therapy may be administered before, during, after, or in various combinations relative to an additional cancer therapy, such as immune checkpoint therapy. The administrations may be in intervals ranging from concurrently to minutes to days to weeks. In some embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an immune cell therapy is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above,

3. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

4. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (AZAR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Publication Nos. 20140294898, 2014022021, and 20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

5. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

6. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### V. ARTICLES OF MANUFACTURE OR KITS

An article of manufacture or a kit is provided comprising immune cells is also provided herein. The article of manufacture or kit can further comprise a package insert comprising instructions for using the immune cells to treat or delay progression of cancer in an individual or to enhance immune function of an individual having cancer. Any of the antigen-specific immune cells described herein may be included in the article of manufacture or kits. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more other agents (e.g., a chemotherapeutic agent, and anti-neoplastic agent). Suitable containers for the one or more agent include, for example, bottles, vials, bags and syringes.

### VI. EXAMPLES

The following Examples section provides further details regarding examples of various embodiments. It should be appreciated by those of skill in the art that the techniques disclosed in the examples that follow represent techniques and/or compositions discovered by the inventors to function well. However, those of skill in the art should, in light of the present disclosure, appreciate that many changes can be made in the specific embodiments which are disclosed and still obtain a like or similar result without departing from the spirit and scope of the disclosure. These examples are illustrations of the methods and systems described herein and are not intended to limit the scope of the disclosure. Non-limiting examples of such include but are not limited to those presented below.

### Example 1

The inventors prospectively characterized peripheral blood and lymphatic fluid T cells from 25 pediatric patients receiving therapeutic thoracic duct access procedures and identified the phenotype and CAR T cell potential. Patients were treated at the Children's Hospital of Philadelphia for congenital or acquired lymphatic malformations or chylothorax. An interventional radiologic procedure in which the thoracic duct is cannulated was performed for therapeutic purposes. Excess fluid as well as a concomitant peripheral blood sample was collected and de-identified under an IRB approved protocol. The inventors quantified the CD3+ population using flow cytometry and expanded these T cells using CD3/CD28 stimulatory beads as in CAR T cell manufacturing. They found an average cell concentration of 1.48e6 cells/mL of lymphatic fluid, with an average of 44% CD3+ T cells versus 18% in blood. The lymphatic fluid to blood percent of Naïve T cells was 36% v. 17%, Stem Central Memory 13% v. 7%, Central Memory 14% v. 27%, Effector Memory 7% v. 5% and Terminal Effector 30% vs 43%. This bias towards Naïve and SCM T cells was correlated with fewer negative checkpoint regulators (PD-1, LAG3 and Tim3) in lymphatic fluid T cells (7%) versus blood (30%). The T cells from lymphatic fluid expanded more robustly to CD3/28 stimulation as in CART manufacture, with an average of 32-fold expansion versus 9-fold for blood. In a xenograft model of pediatric leukemia, the CAR T cells derived from lymphatic fluid produced a deeper and more sustained remission than matched CAR T cells made from peripheral blood. This is the most detailed description of T cells from the lymphatic fluid of children to date. The shift in Naïve and SCM T cells in lymphatic fluid versus peripheral blood of the same patient is a confirmation of expectations. The fact that lymphatic fluid is rich in cells that are potentially better suited to CAR manufacture raises the possibility of collecting cells from this source if peripheral blood is not suitable.

### Example 2

Immunotherapy holds great promise for treatment of cancer resistant to conventional modalities. CAR-T are among the most powerful of these therapies and have shown great activity in pediatric leukemia but not yet in other cancers especially solid tumors. The immune suppressive solid tumor microenvironment appears to influence the circulating T cell profile, with a shift towards T cell phenotypes that are poorly suited to CAR manufacture (see Framework). CART does not work for all patients with leukemia, sometimes due to short persistence and others due to antigen escape.

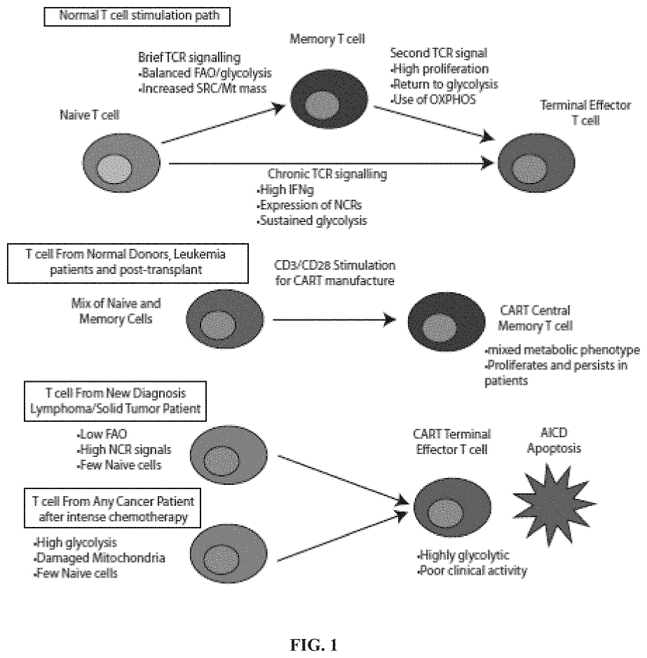

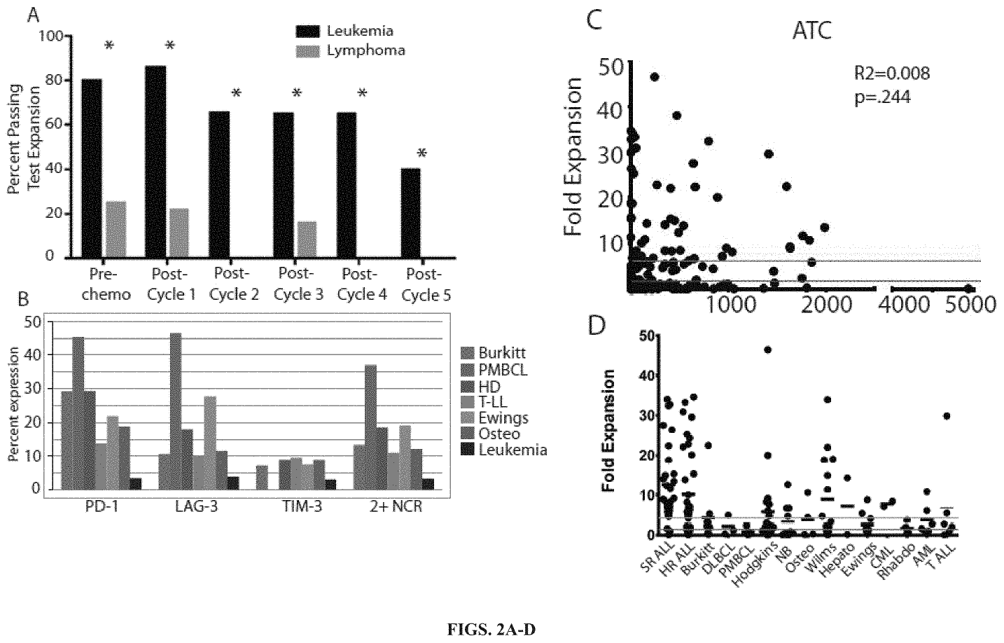

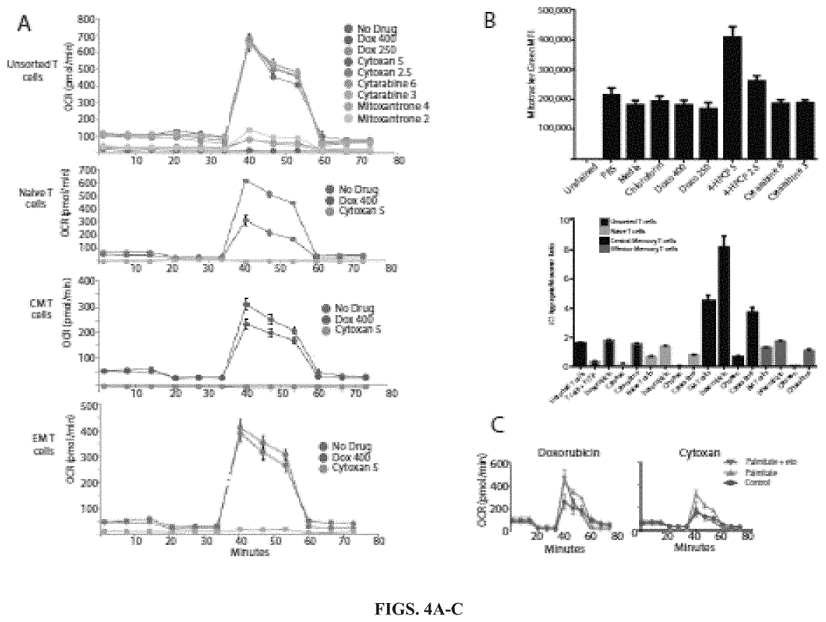

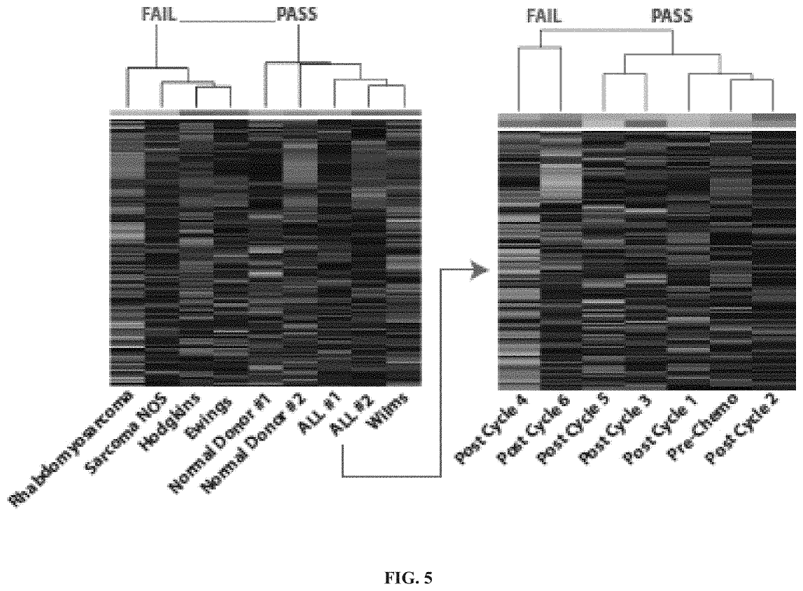

The inventors sought to investigate potential T cell intrinsic mechanisms behind poor CART potenetial in leukemia and potentially solid tumors. They collected T cells from 4 mL of peripheral blood at diagnosis (before therapy) and after every cycle of chemotherapy for 157 patients diagnosed at the Children's Hospital of Philadelphia from 2014-2017. Diagnoses included leukemia, Wilms, osteosarcoma, Ewings, neuroblastoma, rhabdomyosarcoma, Hodgkins and Non-Hodgkins lymphoma and other rare tumors. T cells were surface phenotyped by flow cytometry, tested for CAR manufacture potential by exposure to CD3/28 beads, and analyzed for mitochondrial function and reserve by oxygen consumption analysis (OCR). In addition, the inventors took T cells from normal donors, sorted into T cell subsets and exposed them to chemotherapy alone and in combinations. They characterized the metabolic consequences by Nanostring RNA analysis, metabolite consumption and examination of mitochondrial function. The results are shown in FIGS. 1-5.

Pediatric ALL has seen the most success with CAR T cell therapy, and here the inventors observed that they may have T cells uniquely well suited to this manufacture. Chemotherapy is particularly devastating to Naive T cells, at least in part due to effects on metabolism and mitochondrial function in surviving T cells. Even prior to therapy, T cells from pediatric solid tumor and lymphoma patients show a significant shift in metabolic pathways from normal donors or ALL patients.

### Example 3—Materials and Methods

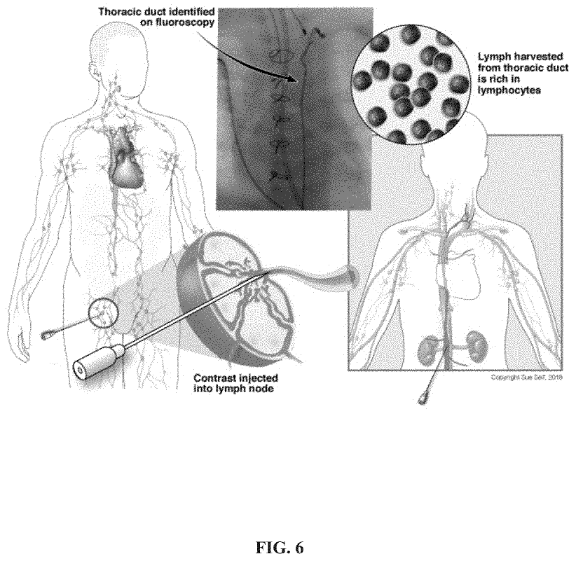

Thoracic duct cannulation. All studies were completed under IRB approval at the Children's Hospital of Philadelphia. Patient samples were obtained from pediatric patients with cardiac defects and lymphatic anomalies who were undergoing thoracic duct cannulation as part of routine clinical care. Informed consent was obtained for all patients. To access the thoracic duct, interventional cardiologists located the thoracic duct as previously described (Nadolski & Itkin, 2012) (FIG. 6). Briefly, under ultrasound guidance, inguinal lymph nodes were identified and injected with 5 mL of oil-based contrast agent (Ethiodol; Savage Laboratories, NDC 0281-7062-37). Using fluoroscopy, the lymphatic channel was traced back to the thoracic duct and a Chiba needle was inserted into the abdomen to access the thoracic duct. A guidewire was then threaded through the Chiba needle, so that a microcatheter could be introduced to collect lymphatic fluid. Lymphatic fluid was then evaluated for T cell studies as described below.

T cell isolation and expansion. Lymphatic fluid was initially measured and then spun at 1200 rpm for 5 minutes. Lysis of any red blood cells, if present, was completed by incubation with tris-buffered ammonium chloride for 5 minutes. Cells were then resuspended in RPMI media containing 10% fetal bovine serum. Blood obtained from patients was combined with Ficol (GE Healthcare 17-1440-02) and centrifuged to separate buffy coat. White blood cells were then removed and resuspended in RPMI containing 10% FBS. Cell counts were completed using a Beckman-Coulter counter. For expansion studies, T cells were incubated with CD3/CD28 Dynabeads (Gibco 11161D) at a ratio of 3:1 for 7 days and counted on day 14. For experiments using cytokine stimulation in addition to beads, 25 ng/mL of interleukin (IL)-7 (R & D systems 207-IL) and 10 ng/mL of IL-15 (R & D systems 247-ILB-025) were added to the media as previously described (Singh et al., 2016).

Flow cytometry. T cells were evaluated for phenotype as previously described using flow cytometry detection of CCR7 FITC (BD Pharmingen 561271), CD62L PE/Cy7 (Biolegend 104417), CD45Ro APC (BD Pharmingen 559865) and CD95 PE (BD Pharmingen 555674).(7) T cell phenotypes were determined using the following staining pattern: T_{N}-CCR7⁺, CD62L⁺, CD45RO⁻, CD95⁻; T_{SCM}-CCR7⁺, CD62L⁺, CD45RO⁻, CD95⁺; T_{CM}-CCR7⁺, CD62L⁺, CD45RO⁺, CD95⁺; T_{EM}-CCRT, CD62L⁻, CD45RO⁺, CD95⁺; and T_{Eff}-CCRT, CD62L⁻, CD45RO⁻, CD95⁺. T cells were evaluated for negative checkpoint regulators using CD279 (PD-1) BV510 (BD Biosciences), CD223 (LAG-3) PerCP-eFluor 710 (eBioscience), and CD366 (TIM-3) BV421 (BD Biosciences), as well as CD4 APC (BD Biosciences 555349) and CD8 FITC (BD Biosciences 347313). CAR expression was evaluated using Biotin-SP Goat Anti-Mouse IgG, F(ab′)₂ primary antibody

(Jackson Immunoresearch 115-065-072) with streptavidin PE secondary (BD Pharmingen 554061), or CD19 CAR isotype primary antibody (gift of Novartis) with Alexa Fluor 647 F(ab′)₂ Fragment Goat Anti-Human IgG secondary (Jackson Immunoresearch 109-606-170). Flow cytometry acquisition was performed with a BD FACSVerse (BD Biosciences). Analysis was completed using FlowJo (Treestar Inc).

Generation of CAR T cells. Lentivirus encoding CD19-directed CAR with CD3ζ and 4-1BB co-stimulatory domains were created using previously described methods (Carpenito et al., 2009). Isolated T cells from blood or lymphatic fluid of the same patient were incubated with lentivirus as previously described (Carpenito et al., 2009), and CAR expression was confirmed using flow cytometry.

Murine xenograft studies. All studies were performed under an approved protocol at The Children's Hospital of Philadelphia. Briefly, 6 to 10-week-old NOD-SCID-γc^{-/-} (NSG) mice bred in house and maintained under pathogen-free conditions were injected via tail vein with 1×10⁶ primary leukemia cells transformed with luciferase (CHP101) in 0.1 ml of sterile PBS, as previously described (Barrett et al., 2011). After achieving appropriate leukemic burden, mice were treated with a single dose of CAR T cells. Each group contained 3-5 mice per arm, for a total of four groups: 1×10⁶ CD19 CAR T cells generated from blood, 1×10⁶ CD19 CAR T cells generated from lymphatic fluid, 1×10⁵ CD19 CAR T cells generated from lymphatic fluid, and 1×10⁶ untransduced T cell control. Mice were monitored frequently for leukemic burden using bioluminescent imaging as previously described (Barrett et al., 2011).

Statistical analysis. Statistical analysis was completed using GraphPad Prism 7 (GraphPad software). Comparison between lymphatic fluid and blood characterization parameters including flow cytometry utilized t tests and 2-way ANOVA. Comparison of bioluminescence for in vivo studies utilized 2-way ANOVA.

### Example 4—Results

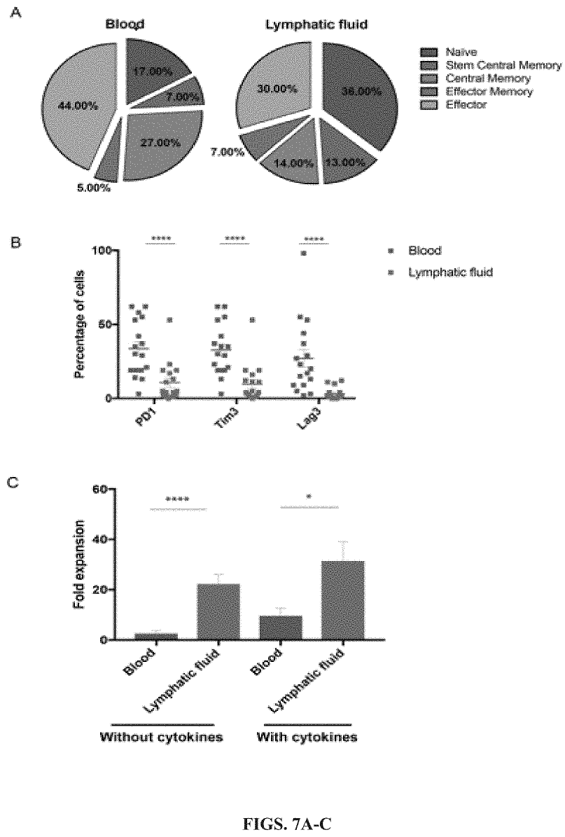

Lymphatic fluid contains higher T cell content with early T cell phenotypes. Cellular content of the lymphatic fluid ranged from 1.5×10⁴ cells/mL to 1.13×10⁷ cells/mL, with a median concentration of 1.11×10⁶ cells/mL (n=25). Lymphatic fluid and paired patient blood (when available) were evaluated for the percentage of total T cells using flow cytometry for CD 4 and CD8. Lymphatic fluid showed higher overall T cell content with 33% T cells (n=16) compared to blood with 17% (n=15) (p=0.0398), (Table 1). Evaluation of the T cell subpopulations revealed a preponderance of early phenotypes in the lymphatic fluid compared to blood (FIG. 7A). Lymphatic fluid contained higher naïve T cells (T_{N}) at 31% and stem central memory (T_{SCM}) at 14%, compared to 15% T_{N} and 8% T_{SCM} in blood (p=0.003 and 0.0271, respectively). Blood contained higher central memory T cells (T_{CM}) at 26% compared to 15% in lymphatic fluid (p=0.0488). There was a trend toward lower effector T cells (T_{Eff}) in lymphatic fluid with 29% compared to 41% T_{Eff} in blood (p=0.0541). Consistent with this shift in T cell phenotype to T_{N} and T_{SCM} in lymphatic fluid, the average size of T cells was much larger. Maximum cell size averaged 773.3 pL in lymphatic fluid (n=22) compared to 445.3 pL in blood (n=19) (Table 1, p<0.0001).

T cells from lymphatic fluid show decreased negative checkpoint regulator expression and increased expansion capacity. The inventors next evaluated T cells for negative checkpoint regulators PD1, Lag3 and Tim3. T cells from blood consistently revealed higher expression of negative checkpoint regulators (FIG. 7B, Table 1). Mean percentage of cells in blood expressing PD1, Lag3 and Tim3 was 33.59%, 32.77%, 27.00% respectively, compared to 10.59%, 9.65% and 3.12% in lymphatic fluid (p<0.0001). T cells were then evaluated for ability to expand after stimulation with beads coated in CD3 and CD28 agonist antibodies, as is performed in our clinical test expansions and good manufacturing practice (GMP) CAR T cell manufacturing process (Levine et al., 1997). Lymphatic fluid T cells expanded on average 22.29-fold, compared to 2.647-fold for T cells from blood (p<0.0001) (FIG. 7C). As the inventors have previously described, cytokines IL-7 and IL-15 can restore expansion capabilities of T cells when added to media in vitro, so the inventors next investigated expansion capabilities of T cells with the addition of IL-7 and IL-15. While the addition of cytokines did improve the expansion of T cells from blood to an average of 9.623-fold, this remained significantly lower than the expansion of T cells from lymphatic fluid averaging 31.38-fold (v0.0194) (FIG. 7C).

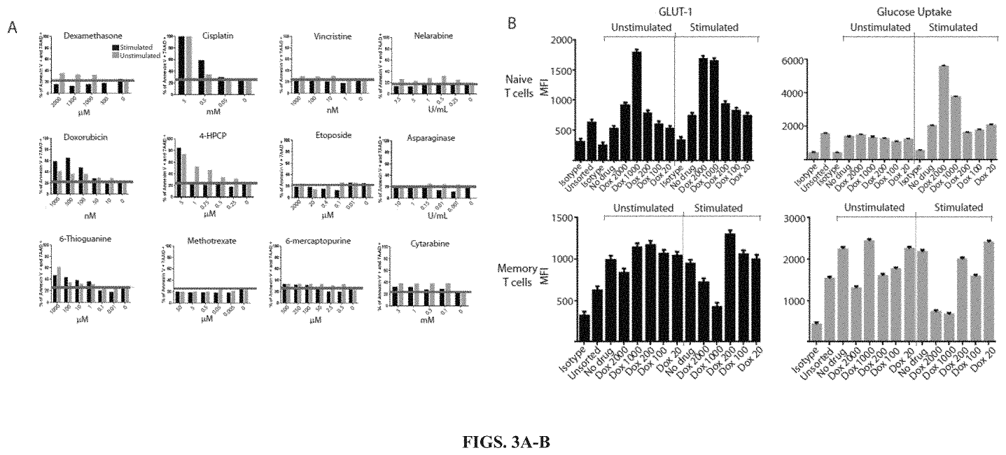

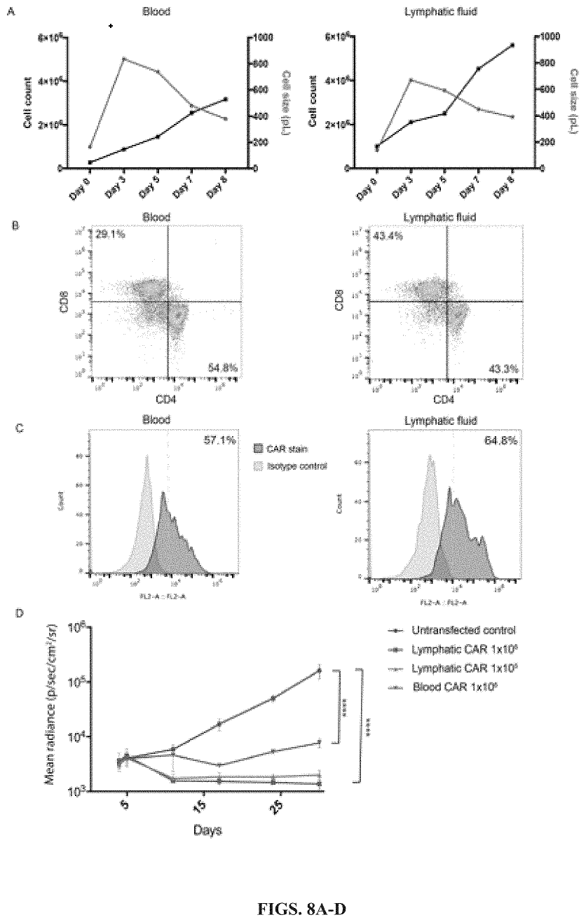

CAR T cells created from lymphatic fluid show similar phenotype to those created from blood. Using paired blood and lymphatic fluid from patients, the inventors generated CD19-directed CAR T cells using lentiviral transduction. T cells from both blood and lymphatic fluid showed similar expansion curves and changes in cell size after lentivirus was introduced (FIG. 8A). Cell size rose in both groups to a peak of 700-900 pL and then rested down to 400 pL range. Cell expansion was also similar in both groups ranging from 5.6 to 11.3-fold expansion across repeated experiments. Following transduction, T cells were evaluated for subpopulations and CAR expression on the surface. Both groups showed a distribution between CD4 and CD8 T cells, with a slight shift toward increased CD4 in CAR T cells made from blood (FIG. 8B). CAR expression on the surface measured by flow cytometry was also similar across both groups at 57.1% and 64.8% (FIG. 3C).

CAR T cell therapy generated from lymphatic fluid produces robust cytotoxicity in vivo. To evaluate CAR efficacy against CD19 positive leukemia, the inventors then created a mouse model of B cell acute lymphoblastic leukemia for comparison of CAR T cells generated from lymphatic fluid and those generated from blood. Mice treated with untransduced T cell controls showed steady leukemic growth over the course of one month, with mean radiance 161480 p/sec/cm²/sr at day 30 (FIG. 8D). Mice treated with a dose of 1×10⁶ CAR T cells generated from blood stabilized the leukemia burden at its baseline with only small increase to mean radiance of 7790 p/sec/cm²/sr at day 30, (p<0.0001) (FIG. 8D). Mice treated with 1×10⁶ CAR T cells generated from lymphatic fluid showed resolution of leukemia with mean radiance 1380 p/sec/cm²/sr (P<0.0001), and even those treated with sub-optimal dose of 1×10⁵ CAR T cells generated from lymphatic fluid similarly eradicated the leukemia with mean radiance 2017 p/sec/cm²/sr at day 30 (P<0.0001) (FIG. 8D).

TABLE 1

| | Lymphatic fluid | Blood | p-value |
|---|---|---|---|
| Percent T cell | 33% | 17% | 0.0398 |
| Maximum T cell size | 773 (474-1159) | 445 (189-892) | <0.0001 |
| (pL) | | | |
| Percent with Negative | 7% | 30% | <0.0001 |
| checkpoint regulators | | | |

### Example 5—Discussion

Here, the inventors describe the first analysis of thoracic duct fluid composition from children. Similar to adults, the pediatric lymphatic fluid was rich in lymphocytes and showed a preponderance of early memory phenotype T cells. The thoracic duct acts as a conduit between lymphatic nodes and blood vessels, where T cells circulate back and forth between blood and lymphatic fluid in pursuit of an activating antigen. Thus, it is not surprising that the total number of T cells in the thoracic duct was high, and a large number of naïve T cells were also present, as naïve T cells have not yet encountered a specific antigen and are re-circulating.

The inventors further showed that T cells isolated from the thoracic duct harbored decreased negative checkpoint regulators and expanded more readily under CAR T manufacturing conditions. T cells from lymphatic fluid were successfully created into CD19 directed CAR T cells, where they showed superior cytotoxicity against leukemia in murine models, compared to CAR T cells generated from blood. This work opens an alternative avenue for T cell collection in adoptive cellular therapy, where the quality and the quantity of cells is paramount. Our group has recently shown that pediatric patients with cancer have a heterogeneous T cell population in the blood, often skewed toward T_{Eff} cells (Das et al., 2019). This is particularly true for patients with solid tumors, as well as all patients treated with chemotherapy (Das et al., 2019). Utilizing T cells from a source rich in naïve and stem central memory cells may be a relatively easy way to improve manufacturing failures as well as augment T cell cytotoxicity. As adoptive cellular therapy takes on a more prominent role where it will be moving into upfront therapy for some pediatric acute lymphoblastic leukemias (upcoming Children's Oncology Group study AALL1721) and new studies emerging in relapsed pediatric solid and brain tumors (such as NCT03500991, NCT03618381, NCT02932956 and NCT02311621), using ideal T cell populations will be come only more important for successful translation of these therapies.

Limitations of this study include that CAR T cells were manufactured from T cells of patients without cancer. It remains unclear if tumor microenvironment will affect T cell populations in the thoracic duct. Future studies will evaluate the T cell composition of patients with cancer and undergoing chemotherapy. In addition, due to limited available samples for this pilot study, large in vivo studies could not be performed or replicated. Future studies will look prospectively at collecting thoracic duct lymphatic fluid and large-scale creation of CART cells.

Leukapheresis is not without safety issues, in particular for small children who cannot tolerate the fluid shifts and large volume of collection needed. In experienced hands, collection of thoracic duct fluid may be equally safe and could result in a far superior cellular therapeutic. This study provides the rationale for alternative collection methods to ensure optimal cellular therapy products for pediatric patients with cancer.

All of the methods disclosed and claimed herein can be made and executed without undue experimentation in light of the present disclosure. While the compositions and methods of this disclosure have been described in terms of particular embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. REFERENCES

The following references, to the extent that they provide exemplary procedural or other details supplementary to those set forth herein, are specifically incorporated herein by reference.
Austin-Ward and Villaseca, Revista Medica de Chile, 126(7):838-845, 1998.
Ausubel et al., CURRENT PROTOCOLS IN MOLECULAR BIOLOGY, Greene Publishing Associates and John Wiley & Sons, N Y, 1994.
Azuma et al., Cell Immunol., 116(1):123-134, 1988.
Barrett et al., Blood, 118(15):e112-7 doi 10.1182/blood-2011-04-346528, 2011.
Bisht et al., Indian J. Cancer, 47(4):443-451, 2010.
Boussif et al., Proc. Nat'l Acad. Sci. USA, 92:7297-7301, 1995.
Bukowski et al., Clin. Cancer Res., 4(10):2337-2347, 1998.
Camacho et al., J. Clin. Oncology 22(145): Abstract No. 2505, 2004.
Carpenito et al., Proc. Nat'l Acad. Sci. USA, 106(9):3360-5 doi 10.1073/pnas.0813101106, 2009.
Christodoulides et al., Microbiology, 144(Pt 11):3027-3037, 1998.
Das et al., Cancer Discovery, CD-18-1314 doi 10.1158/2159-8290.Cd-18-1314, 2019.
Davidson et al., J. Immunother., 21(5):389-398, 1998.
Fong et al., J. Immunol. 166:4254-4259, 2001.
Ford et al., Gene Therapy 8:1-4, 2001.
Gupta and Kanodia, Nat'l Med. J. India, 15(4):202-207, 2002.
Hanibuchi et al., Int. J. Cancer, 78(4):480-485, 1998.
Heemskerk et al., Human Gene Ther. 19:496-510 (2008).
Hellstrand et al., Acta Oncologica, 37(4):347-353, 1998.
Hermanson and Kaufman, Front. Immunol., 6, 195, 2015.
Hollander, Front. Immun., 3:3, 2012.
Hui and Hashimoto, Infection Immun., 66(11):5329-5336, 1998.
Hurwitz et al., Proc. Nat'l Acad. Sci. USA 95(17): 10067-10071, 1998.
Husson et al., J. Bacteriol., 172(2):519-524, 1990.
Jacobs et al., Nature, 327(6122):532-535, 1987.
Johnson et al., Blood 114:535-46 (2009).
Levine et al., J. Immunol., 159(12):5921-30, 1997.
Lieberman, Pharmaceutical Dosage Forms (vols. 1-3, 1992)
Lloyd, The Art, Science and Technology of Pharmaceutical Compounding (1999)
Late et al., Adv. Tuberc. Res., 21:107-93; 194-245, 1984.
Luelmo, Am. Rev. Respir. Dis., 125(3 Pt 2):70-72, 1982.
Martin et al., Nature, 345(6277):739-743, 1990.
Mokyr et al., Cancer Res. 58:5301-5304, 1998.
Nadolski & Itkin, J., Vascular Interventional Radiol., 23(5):613-6, 2012.
Pardoll, Nat. Rev. Cancer, 12(4): 252-64, 2012b.
Pickar, Dosage Calculations, 1999.
Prochiantz, Nat. Methods 4:119-20, 2007.
Qin et al., Proc. Nat'l Acad. Sci. USA, 95(24):14411-14416, 1998.
Rabinovich et al., Science, 265(5177):1401-1404, 1994.
Remington: The Science and Practice of Pharmacy, 20th Edition, 2003, Gennaro, Ed.,
Lippincott, Williams & Wilkins.
Remington's Pharmaceutical Sciences, 15th ed., pages 1035-1038 and 1570-1580, Mack Publishing Company, Easton, Pa., 1980.
Sambrook et al., MOLECULAR CLONING, A LABORATORY MANUAL, Cold Springs Harbor Press (Cold Springs Harbor, N Y 1989).
Schonfeld et al. Mol. Ther., 23, 330-338, 2015.
Schumacher and Schreiber, Science, 348:69-74, 2015.
Shah et al., PLoS One, 8:e776781, 2013.
Singh et al., Sci. Transl. Med.; 8(320):320ra3 doi 10.1126/scitranslmed.aad5222, 2016.
Singleton et al., DICTIONARY OF MICROBIOLOGY AND MOLECULAR BIOLOGY, 2nd ed., J. Wiley & Sons (New York, N.Y. 1994).
Snapper et al., Proc. Nat'l Acad. Sci. USA, 85(18):6987-6991, 1988.
Spanholtz et al., PLoS One, 6(6):e20740, 2011.
Takada et al., J. Clin. Microbiol 33(3):658-660, 1995.
Terakura et al., Blood. 1:72-82, 2012.
U.S. Pat. No. 4,435,386
U.S. Pat. No. 4,436,727
U.S. Pat. No. 4,436,728
U.S. Pat. No. 4,505,899
U.S. Pat. No. 4,505,900
U.S. Pat. No. 4,520,019
U.S. Pat. No. 4,579,945
U.S. Pat. No. 4,866,034
U.S. Pat. No. 4,877,611
U.S. Pat. No. 4,950,645
U.S. Pat. No. 5,739,169
U.S. Pat. No. 5,801,005
U.S. Pat. No. 5,824,311
U.S. Pat. No. 5,830,880
U.S. Pat. No. 5,846,945
U.S. Pat. No. 6,207,156
U.S. Pat. No. 8,008,449
U.S. Pat. No. 8,017,114
U.S. Pat. No. 8,119,129
U.S. Pat. No. 8,329,867
U.S. Pat. No. 8,354,509
U.S. Pat. No. 8,735,553
Wang et al., J. Immunother. 35(9):689-701, 2012.
WO 00/37504
WO 01/14424
WO 91/16347
WO 98/42752
WO1995001994
WO1998042752
WO2000037504
WO2001014424
WO2009/101611
WO2009/114335
WO2010/027827
WO2011/066342
WO2015016718
Yamamoto et al., Jpn. J Cancer Res., 79:866 873, 1988.
Yin et al., J. Biol. Resp. Modif., 8:190 205, 1989.

1. A method of preparing a T cell comprising:
(a) obtaining a T cell-containing sample from the lymphatic system of a subject;
(b) isolating a T cell subpopulation from said sample; and
(c) generating a chimeric antigen receptor (CAR) T cell from the isolated T cell subpopulation of step (b).

2. The method of claim 1, wherein step (b) comprises isolating a cell based on CD3/28 expression.

3. The method of claim 1, wherein the T cell subpopulation isolated in step (b) has reduced levels of negative checkpoint regulators (NCR) as compared to the average T cell NCR isolated from peripheral blood of said subject, and/or has increased expression of GLUT-1 as compared to the average T cell in said sample.

4. The method of claim 3, wherein the NCR is PD-1, LAG3 or Tim3.

5. The method of claim 1, wherein the T cell subpopulation isolated in step (b) is enhanced in Naïve T cell and/or a Stem Cell Memory (SCM) T cell content as compared to an unisolated population.

6. The method of claim 1, wherein said subject is a disease-free subject.

7. The method of claim 1, wherein said subject is diseased but does not have cancer.

8. The method of claim 7, wherein said subject has a congenital or acquired lymphatic malformation or chylothorax.

9. The method of claim 1, wherein said subject has cancer.

10. The method of claim 9, wherein said cancer is a solid cancer such as lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, brain cancer, thyroid cancer, various types of head and neck cancer, or melanoma.

11. The method of claim 9, wherein said cancer is a blood cancer such as leukemia or lymphoma.

12. The method of claim 9, wherein said subject has not been subjected to chemotherapy or radiotherapy.

13. The method of claim 9, wherein said subject has been subjected to chemotherapy or radiotherapy.

14. The method of claim 9, wherein said subject has been subjected to chemotherapy and radiotherapy.

15. The method of claim 1, wherein said subject is a human subject.

16. A method of treating cancer in a human subject in need thereof comprising administering to the subject an effective amount of a cell therapy comprising one or more cells produced in accordance with claim 1.

17. The method of claim 16, further comprising administering to said human subject a second cancer therapy.

18. The method of claim 17, wherein said second cancer therapy is chemotherapy, immunotherapy, radiotherapy, hormone therapy or surgery.

19. The method of claim 17, wherein said second cancer therapy is administered at the same time as the cell therapy.

20. The method of claim 17, wherein said second cancer therapy is administered before or after the cell therapy.

21. The method of claim 16, further comprising administering to said human subject a second administration of an effective amount of said one or more cells.

22. The method of claim 16, wherein said cancer is a metastatic, recurrent or drug-resistant cancer.

23. The method of claim 16, wherein said cell therapy is administered local to cancer site, region to a cancer site, or systemically.

24. The method of claim 16, wherein said cancer is solid cancer such as lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, brain cancer, vulval cancer, thyroid cancer, various types of head and neck cancer, and melanoma.

25. The method of claim 16, wherein said cancer is a blood cancer such as leukemia or lymphoma.

26. A vaccine composition comprising a cell produced according to claim 1.

27. The vaccine composition of claim 26, further comprising an adjuvant.

28. The vaccine composition of claim 26, further comprising a biological response modifier.

29. The vaccine composition of claim 26, further comprising a chemokine.

30. The vaccine composition of claim 26, wherein two distinct CAR T cells with different binding specificities are comprised in said vaccine composition.

31. A method of generating an anti-cancer immune response is a subject comprising administering to said subject a vaccine composition according to claim 26.


SEQUENCE LISTING (10 sequences)

SEQ ID NO: 1 - protein, 21 aa, Artificial sequence
  Synthetic amino acid
IYIWAPLAGTCGVLLLSLVIT

SEQ ID NO: 2 - DNA, 63 bp, Artificial sequence
  Synthetic oligonucleotide
atctacatctgggctccactggcaggaacctgtggcgtgctgctgctgtccctggtcatcaca

SEQ ID NO: 3 - protein, 27 aa, Artificial sequence
  Synthetic amino acid
FWVLVVVGGVLACYSLLVTVAFIIFWV

SEQ ID NO: 4 - DNA, 81 bp, Artificial sequence
  Synthetic oligonucleotide
ttttgggtgctggtggtggttggtggagtcctggcttgctatagcttgctagtaacagtggcctttattattttctgggtg

SEQ ID NO: 5 - DNA, 339 bp, Artificial sequence
  Synthetic oligonucleotide
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 6 - protein, 41 aa, Artificial sequence
  Synthetic amino acid
RSKRSRLLHSDYMNMTPRRPGPTRKHYQPYAPPRDFAAYRS

SEQ ID NO: 7 - DNA, 123 bp, Artificial sequence
  Synthetic oligonucleotide
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 8 - protein, 42 aa, Artificial sequence
  Synthetic amino acid
KRGRKKLLYIFKQPFMRPVQTTQEEDGCSCRFPEEEEGGCEL

SEQ ID NO: 9 - DNA, 126 bp, Artificial sequence
  Synthetic oligonucleotide
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 10 - protein, 15 aa, Artificial sequence
  Synthetic amino acid
GGGGSGGGGSGGGGS